(19) United States
(12) Patent Application Publication
(10) Pub. No.: US 2024/0389847 A1
(43) Pub. Date: Nov. 28, 2024

(54) IMAGE PROCESSING DEVICE, TREATMENT SYSTEM, AND IMAGE PROCESSING METHOD

(71) Applicant: Olympus Corporation, Tokyo (JP)

(72) Inventors: Hiroshi Suzuki, Tokyo (JP); Koichiro Watanabe, Tokyo (JP)

(21) Appl. No.: 18/790,340

(22) Filed: Jul. 31, 2024

Related U.S. Application Data
(63) Continuation of application No. PCT/JP2022/009563, filed on Mar. 4, 2022.

(51) Int. Cl. A61B 1/317; A61B 1/045

(52) U.S. Cl. CPC A61B 1/317; A61B 1/045

(57) ABSTRACT

An image processing device includes a processor including hardware. The processor is configured to: acquire first image data partially including a region in which a living body is treated with at least an energy treatment instrument; detect a change in tone from at least a part of a region of a first image corresponding to the first image data to obtain a first detection result; perform tone correction on the first image based on the first detection result to generate first correction-image data; and generate a display image based on the first correction-image data.

### CROSS-REFERENCE TO RELATED APPLICATION

This application is a continuation of International Application No. PCT/JP2022/009563, filed on Mar. 4, 2022, the entire contents of which are incorporated herein by reference.

### BACKGROUND

### 1. Technical Field

The present disclosure relates to an image processing device, a treatment system, and an image processing method.

### 2. Related Art

In arthroscopic surgery, a technique that uses an irrigation device to inflate the inside of a joint with irrigation fluid, such as physiological saline solution, to secure a field of view and perform a procedure on a treatment site has been known (for example, Japanese Patent No. 4564595). In this technique, because bone powder, which is scrapings of bone, and marrow fluid are generated by crushing a bone with a hammering action of an ultrasound treatment instrument, the visibility of a treatment area is ensured by expelling bone powder and marrow fluid from the field of view of an endoscope with irrigation fluid.

### SUMMARY

In some embodiments, an image processing device includes a processor including hardware, the processor being configured to: acquire first image data partially including a region in which a living body is treated with at least an energy treatment instrument; detect a change in tone from at least a part of a region of a first image corresponding to the first image data to obtain a first detection result; perform tone correction on the first image based on the first detection result to generate first correction-image data; and generate a display image based on the first correction-image data.

In some embodiments, an image processing device includes a processor including hardware, the processor being configured to: acquire a first image corresponding to first image data that includes a region in which a living body is treated with an energy treatment instrument; acquire a second image corresponding to second image data having a different wavelength from the first image; detect a change in tone from at least a part of a region of the first image to obtain a detection result; perform tone correction on the second image based on the detection result to generate correction image data; and generate a display image based on the correction image data.

In some embodiments, a treatment system includes: an energy treatment instrument that can be inserted into a subject, and that is capable of treating a treatment target site; an endoscope that can be inserted into the subject, and that is capable of generating first image data by imaging at least the treatment target site; and an image processing device that performs image processing with respect to the first image data to output the processed first image data to a display, the image processing device comprising a processor comprising hardware, the processor being configured to: acquire the first image data; detect a change in tone from at least a part of a region of a first image corresponding to the first image data to obtain a first detection result; perform tone correction on the first image based on the first detection result to generate first correction-image data; and generate a display image based on the first correction-image data.

In some embodiments, provided is an image processing method that is performed by an image processing device included in a processor including hardware. The method includes: acquiring first image data that includes a region in which a living body is treated with an energy treatment instrument; detecting a change in tone from at least a part of a region of a first image corresponding to the first image data to obtain a first detection result; performing tone correction on the first image based on the first detection result to generate first correction-image data; and generating a display image based on the first correction-image data.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of a treatment system according to a first embodiment of the present disclosure;

FIG. 2 is a diagram illustrating a state in which a bone hole is formed by an ultrasound probe according to the first embodiment of the present disclosure;

FIG. 3A is a schematic diagram illustrating a schematic configuration of the ultrasound probe according to the first embodiment of the present disclosure;

FIG. 3B is a schematic diagram illustrating a direction of an arrow A in FIG. 3A;

FIG. 4 is a block diagram illustrating an overview of a functional configuration of the entire treatment system according to the first embodiment of the present disclosure;

FIG. 5 is a block diagram illustrating a detailed functional configuration of an endoscope device according to the first embodiment of the present disclosure;

FIG. 6A is a diagram illustrating a state in which a field of view of the endoscope according to the first embodiment of the present disclosure is in a good condition;

FIG. 6B is a diagram illustrating a state in which a field of view of the endoscope according to the first embodiment of the present disclosure is in a poor condition;

FIG. 7 is a block diagram illustrating a detailed functional configuration of a processing device according to the first embodiment of the present disclosure;

FIG. 8 is a block diagram illustrating a detailed functional configuration of an irrigation device according to the first embodiment of the present disclosure;

FIG. 9 is a block diagram illustrating a detailed functional configuration of an illumination device according to the first embodiment of the present disclosure;

FIG. 10 is a block diagram illustrating a functional configuration of an imaging device according to the first embodiment of the present disclosure;

FIG. 11 is a diagram schematically illustrating a configuration of a pixel unit according to the first embodiment of the present disclosure;

FIG. 12 is a diagram schematically illustrating a configuration of a color filter according to the first embodiment of the present disclosure;

FIG. 13 is a diagram schematically illustrating a sensitivity and a wavelength band of each filter according to the first embodiment of the present disclosure;

FIG. 14 is a block diagram illustrating a detailed functional configuration of an image processing unit according to the first embodiment of the present disclosure;

FIG. 15 is a block diagram illustrating a detailed functional configuration of a first correction-image generating unit according to the first embodiment of the present disclosure;

FIG. 16 is a flowchart explaining an overview of a procedure performed by an operator by using the treatment system according to the first embodiment of the present disclosure;

FIG. 17 is a flowchart explaining about an overview of processing performed in a cutting procedure by an endoscope control device according to the first embodiment of the present disclosure;

FIG. 18 is a flowchart illustrating a detailed overview of turbidity-treatment control processing in FIG. 17;

FIG. 19 is a diagram illustrating an example of temporally continuous first images in the field of view of the endoscope that are generated by a display-image generating unit based on the first image and that are output to a display device when haze correction processing by the first correction-image generating unit according to the first embodiment of the present disclosure has not been performed;

FIG. 20 is a diagram illustrating an example of temporally continuous first correction images in the field of view of the endoscope that are generated by the display-image generating unit based on the first correction image and that are output to the display device when the haze correction processing by the first correction-image generating unit according to the first embodiment of the present disclosure is performed;

FIG. 21 is a diagram illustrating an example of temporally continuous second correction images in the field of view of the endoscope that are generated by the display-image generating unit based on the second correction image and that are output to the display device when edge enhancement processing by a second correction-image generating unit according to the first embodiment of the present disclosure is performed;

FIG. 22 is a diagram illustrating an example of temporally continuous composite images in the field of view of the endoscope that are generated by the display-image generating unit based on the composite image and that are output to the display device when compositing processing by a composite-image generating unit according to the first embodiment of the present disclosure is performed;

FIG. 23 is a diagram illustrating an example of temporally continuous images in the field of view of the endoscope to output the first correction image and the second correction image to the display device by the display-image generating unit according to the first embodiment of the present disclosure;

FIG. 24 is a block diagram illustrating a functional configuration of an endoscope according to a second embodiment of the present disclosure;

FIG. 25 is a diagram illustrating an example of a composite image generated by a composite-image generating unit according to the second embodiment of the present disclosure;

FIG. 26 is a block diagram illustrating a functional configuration of an endoscope according to a third embodiment of the present disclosure;

FIG. 27 is a block diagram illustrating a functional configuration of an illumination device according to the third embodiment of the present disclosure;

FIG. 28 is a schematic diagram illustrating a schematic configuration of an illuminating unit according to the third embodiment of the present disclosure;

FIG. 29 is a diagram illustrating a relationship between transmission characteristics and wavelength bands of a red filter, a green filter, and a blue filter according to the third embodiment of the present disclosure; and

FIG. 30 is a diagram illustrating a relationship between a transmission characteristic and a wavelength band of an IR filter according to the third embodiment of the present disclosure.

### DETAILED DESCRIPTION

Herein after, embodiments to implement the present disclosure will be explained in detail with reference to the drawings. The following embodiments are not intended to limit the present disclosure. Moreover, the respective drawings to be referred to in the following explanation only provide a schematic representation of shape, size, and positional relationship to the extent that the content can be understood. That is, the present disclosure is not limited to the shapes, the sizes, and the positional relationships provided in the respective drawings. Furthermore, in the following explanation, identical reference symbols are assigned to identical parts in description of the drawings.

### First Embodiment

### Schematic Configuration of Treatment System

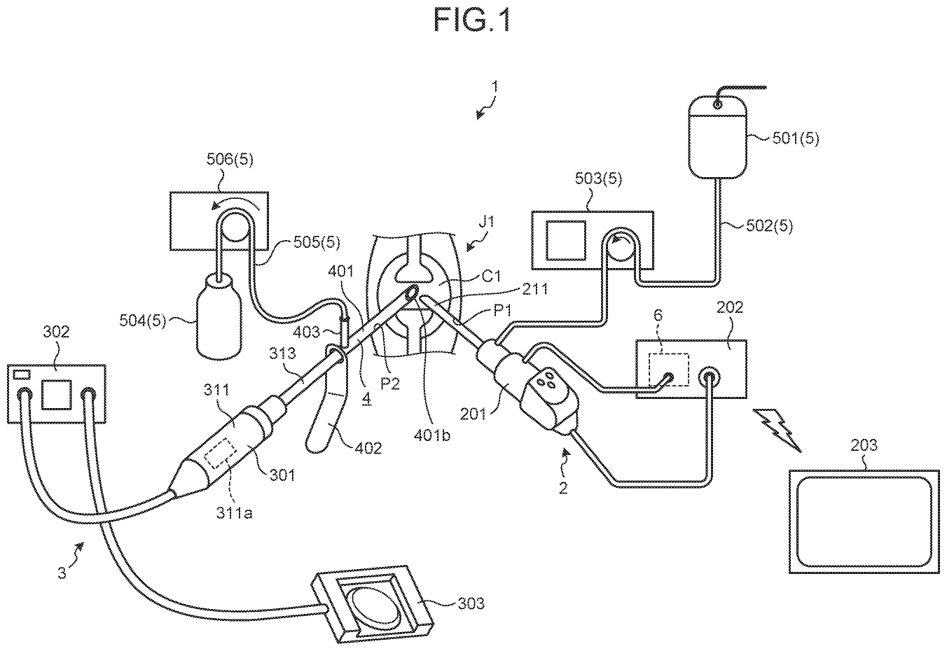

FIG. 1 is a diagram illustrating a schematic configuration of a treatment system 1 according to a first embodiment. The treatment system 1 illustrated in FIG. 1 applies ultrasound vibrations to a living tissue, such as bones, to thereby treat the living tissue. The procedure herein is, for example, removal or excision of bone or the like. In FIG. 1, as the treatment system 1, a treatment system that performs anterior cruciate ligament reconstruction surgery is exemplified.

The treatment system 1 illustrated in FIG. 1 includes an endoscope device 2, a treatment device 3, a guiding device 4, an irrigation device 5, and an illumination device 6.

### Configuration of Endoscope

First, a configuration of the endoscope device 2 will be explained.

The endoscope device 2 includes an endoscope 201, an endoscope control device 202, and a display device 203.

The endoscope 201 has an insertion portion 211, a distal end portion of which is inserted into a joint cavity Cl through a first portal P1 that communicates an inside of the joint cavity Cl of a knee joint J1 of a subject and an outside of a skin. The endoscope 201 illuminates the inside of the joint cavity Cl, captures illumination light (subject image) reflected in the inside of the joint cavity Cl, and images a subject image to generate image data.

The endoscope control device 202 performs various kinds of image processing with respect to the image data captured by the endoscope 201, and displays the image data subjected to the image processing on the display device 203. The endoscope control device 202 is connected to the endoscope 201 and the display device 203 wiredly or wirelessly.

The display device 203 receives data transmitted from respective devices constituting the treatment system 1, image data (display image), sound data, and the like through the endoscope control device 202, and performs display of the display image, notification, and output according to the received data. The display device 203 is constituted of a liquid crystal or organic electro-luminescence (EL) display panel.

### Configuration of Processing Device

Next, a configuration of the treatment device 3 will be explained.

The treatment device 3 includes a treatment instrument 301, a treatment-instrument control device 302, and a foot switch 303.

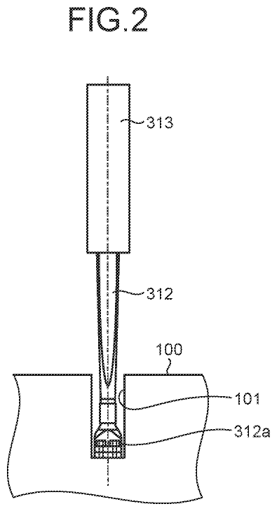

The treatment instrument 301 includes a treatment-instrument main unit 311, an ultrasound probe 312 (refer to FIG. 2 described later), and a sheath 313.

The treatment-instrument main unit 311 is formed in a cylindrical shape. Moreover, inside the treatment-instrument main unit 311, an ultrasound transducer 312a (refer to FIG. 2 described later) that is constituted of a bolt-clamped Langevin-type transducer, and that generates ultrasound vibrations according to a supplied driving power is housed.

The treatment-instrument control device 302 supplies a driving power to the ultrasound transducer 312a according to an operation to the foot switch 303 by an operator. Supply of the driving power is not limited to be performed by operation of the foot switch 303, but may also be performed, for example, according to an operation of an operating unit (not illustrated) provided in the treatment instrument 301.

The foot switch 303 is an input interface for the operator to operate when the ultrasound probe 312 is to be activated.

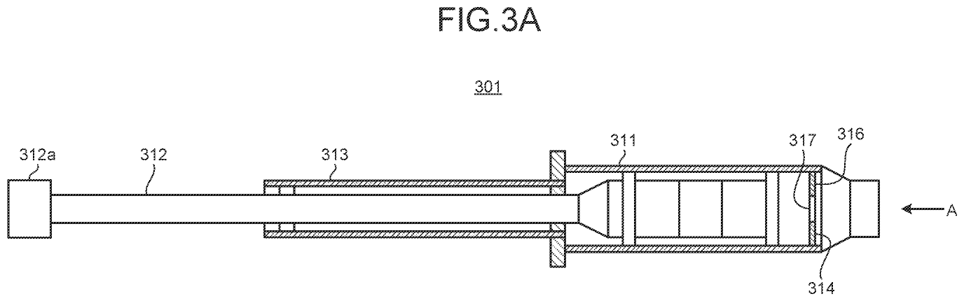

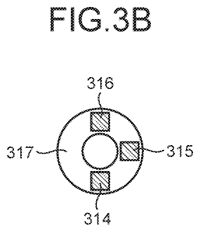

Next, the ultrasound probe 312 will be explained. FIG. 2 is a diagram illustrating a state in which a bone hole 101 is formed by the ultrasound probe 312. FIG. 3A is a schematic diagram illustrating a schematic configuration of the ultrasound probe 312. FIG. 3B is a schematic diagram illustrating a direction of an arrow A in FIG. 3A.

As illustrated in FIG. 2, FIG. 3A, and FIG. 3B, the ultrasound probe 312 is constituted of, for example, titanium alloy or the like, and has a substantially cylindrical shape. Moreover, a proximal end portion of the ultrasound probe 312 is connected to the ultrasound transducer 312a inside the treatment-instrument main unit 311. Furthermore, the ultrasound probe 312 transmits ultrasound vibrations generated by the ultrasound transducer 312a from a proximal end to a distal end. Specifically, ultrasound vibrations in the first embodiment are vertical vibrations along a longitudinal direction of the ultrasound probe 312 (up and down direction in FIG. 2). Moreover, at a distal end portion of the ultrasound probe 312, the ultrasound transducer 312a is provided as illustrated in FIG. 2.

The sheath 313 is formed in a cylindrical shape thinner and longer than the treatment-instrument main unit 311, and covers an outer circumference of the ultrasound probe 312 up to an arbitrary length from the treatment-instrument main unit 311.

The ultrasound transducer 312a of the ultrasound probe 312 in the treatment instrument 301 thus configured is inserted into the joint cavity Cl while being guided by the guiding device 4 inserted into the joint cavity Cl through a second portal P2 that communicates between the inside of the joint cavity Cl and an outside of the skin.

Subsequently, the treatment instrument 301 generates ultrasound vibrations in a state in which the ultrasound transducer 312a of the ultrasound probe 312 is in contact with a treatment target site 100 of a bone. A portion of the bone mechanically impacted with the ultrasound transducer 312a is then crushed into fine particles by hammering action (refer to FIG. 2).

Thereafter, when the ultrasound transducer 312a of the ultrasound probe 312 is pushed against the treatment target site 100 by the operator, the treatment instrument 301 enters into the treatment target site 100 while crashing the bone with the ultrasound transducer 312a. Thus, the bone hole 101 is formed in the treatment target site 100.

Moreover, at an proximal end of the treatment-instrument main unit 311, a circuit board 317 on which a position detecting unit 314, a central processing unit (CPU) 315, a memory 316 are mounted is arranged (refer to FIG. 3A and FIG. 3B).

The position detecting unit 314 includes a sensor that detects rotation or movement of the treatment instrument 301. The position detecting unit 314 detects movement in three axial directions perpendicular to one another, including an axis parallel to a longitudinal axis of the ultrasound probe 312, and rotation about the respective axes. The treatment-instrument control device 302 determines that the treatment instrument 301 is in a still state when detection results of the position detecting unit 314 do not change for a predetermined time. The position detecting unit 314 is constituted of, for example, a tree-axis gyro sensor, acceleration sensor, and the like.

The CPU 315 controls operation of the position detecting unit 314, or transmits and receives information to and from the treatment-instrument control device 302. The CPU 315 loads a program stored in the memory 316 onto a work area of a memory to execute, and controls the respective components and the like through execution of the program by a processor, and hardware and software thereby cooperate to implement a functional module that matches a predetermined purpose.

### Configuration of Guiding Device

Next, a configuration of the guiding device 4 will be explained.

In FIG. 1, the guiding device 4 is inserted in to the joint cavity Cl through the second portal P2, and guides the distal end portion of the ultrasound probe 312 in the treatment instrument 301 to the inside of the joint cavity Cl.

The guiding device 4 includes a guide main unit 401, a handle portion 402, and a cock-equipped drainage unit 403.

The guide main unit 401 has a tubular shape, and has a through hole 401a in which the ultrasound probe 312 is inserted (refer to FIG. 1). The guide main unit 401 controls travel of the ultrasound probe 312 inserted in the through hole 401a to be in a certain direction, and guides the movement of the ultrasound probe 312. In the first embodiment, a cross-sectional shape perpendicular to a center axis on an outer peripheral surface and an inner peripheral surface of the guide main unit 401 is a substantially circular shape. Moreover, the guide main unit 401 is tapered to become thinner toward its distal end. That is, a distal end surface 401b of the guide main unit 401 has an inclined surface that intersects the center axis at an angle.

The cock-equipped drainage unit 403 is arranged on an outer peripheral surface of the guide main unit 401, and has a tubular shape that communicates with an interior of the guide main unit 401. To the cock-equipped drainage unit 403, one end of a drainage tube 505 of the irrigation device 5 is connected, to be a flow channel communicating between the guide main unit 401 and the drainage tube 505 of the irrigation device 5. This flow channel is configured to be openable and closable by operating a cock (not illustrated) arranged in the cock-equipped drainage unit 403.

### Configuration of Irrigation Device

Next, a configuration of the irrigation device 5 will be explained.

In FIG. 1, the irrigation device 5 feeds irrigation fluid, such as sterilized saline solution, to the inside of the joint cavity Cl, and discharges the irrigation fluid to the outside the joint cavity Cl.

The irrigation device 5 includes a liquid source 501, a liquid feeding tube 502, an infusion pump 503, a drainage bottle 504, the drainage tube 505, and a drainage pump 506 (refer to FIG. 1).

The liquid source 501 contains irrigation fluid thereinside. To the liquid source 501, the liquid feeding tube 502 is connected. The irrigation fluid is sterilized saline solution or the like. The liquid source 501 is constituted of, for example, a bottle, or the like.

One end of the liquid feeding tube 502 is connected to the liquid source 501, and the other end thereof is connected to the endoscope 201.

The infusion pump 503 sends out the irrigation fluid toward the endoscope 201 from the liquid source 501 through the liquid feeding tube 502. The irrigation fluid sent to the endoscope 201 is sent to the inside of the joint cavity Cl from a liquid feeding hole formed in a distal end portion of the insertion portion 211.

The drainage bottle 504 contains irrigation fluid that is discharged out from the joint cavity Cl. To the drainage bottle 504, the drainage tube 505 is connected.

One end of the drainage tube 505 is connected to the guiding device 4, and the other end thereof is connected to the drainage bottle 504.

The drainage pump 506 discharges the irrigation fluid inside the joint cavity Cl to the drainage bottle 504 through the flow channel of the drainage tube 505 from the guiding device 4 inserted into the joint cavity Cl. Although the first embodiment is explained using the drainage pump 506, it is not limited thereto, and a suction device that is equipped in the facility may be used.

### Configuration of Illumination Device

Next, a configuration of the illumination device 6 will be explained.

In FIG. 1, the illumination device has two light sources 6 that respectively emit two illumination lights having different wavelengths from each other. The two illumination lights are, for example, a white light that is visible light and an infrared light that is invisible light. The illumination light from the illumination device 6 is propagated to the endoscope 201 through a light guide, and is irradiated from a distal end of the endoscope 201.

### Functional Configuration of Treatment System

Next, a functional configuration of the entire treatment system will be explained.

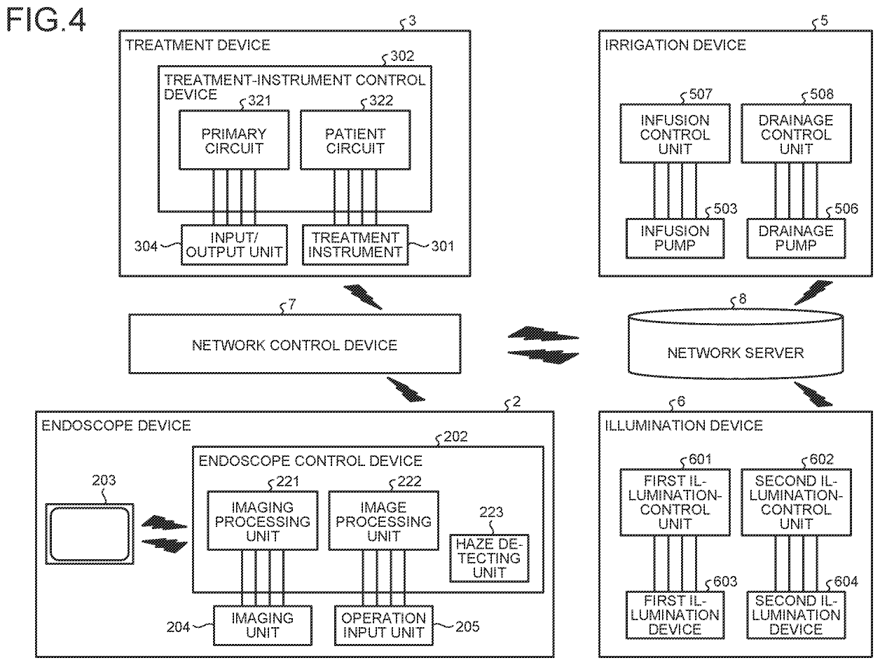

FIG. 4 is a block diagram illustrating an overview of a functional configuration of the entire treatment system 1. The treatment system 1 illustrated in FIG. 4 further includes a network control device 7 that controls communications of the entire system, and a network server 8 that stores various kinds of data, in addition to the configuration described above (refer to FIG. 1).

The network control device 7 is connected to the endoscope device 2, the treatment device 3, the irrigation device 5, the illumination device 6, and the network server 8 in a communication-enabled manner. Although an example in which the devices are connected wirelessly is illustrated in FIG. 4, they may be connected wiredly. In the following, a detailed functional configuration of the endoscope device ″, the treatment device 3, the irrigation device 5, and the illumination device 6 will be explained.

The network server 8 is connected to the endoscope device 2, the treatment device 3, the irrigation device 5, the illumination device 6, and the network control device 7 in a communication-enabled manner. The network server 8 stores various kinds of data of the respective devices that constitute the treatment system 1. The network server 8 is constituted of, for example, a processor having hardware, such as a CPU, and a memory, such as a hard disk drive (HDD) and a solid state device (SSD).

### Functional Configuration of Endoscope Device

Next, a functional configuration of the endoscope device 2 described above will be explained.

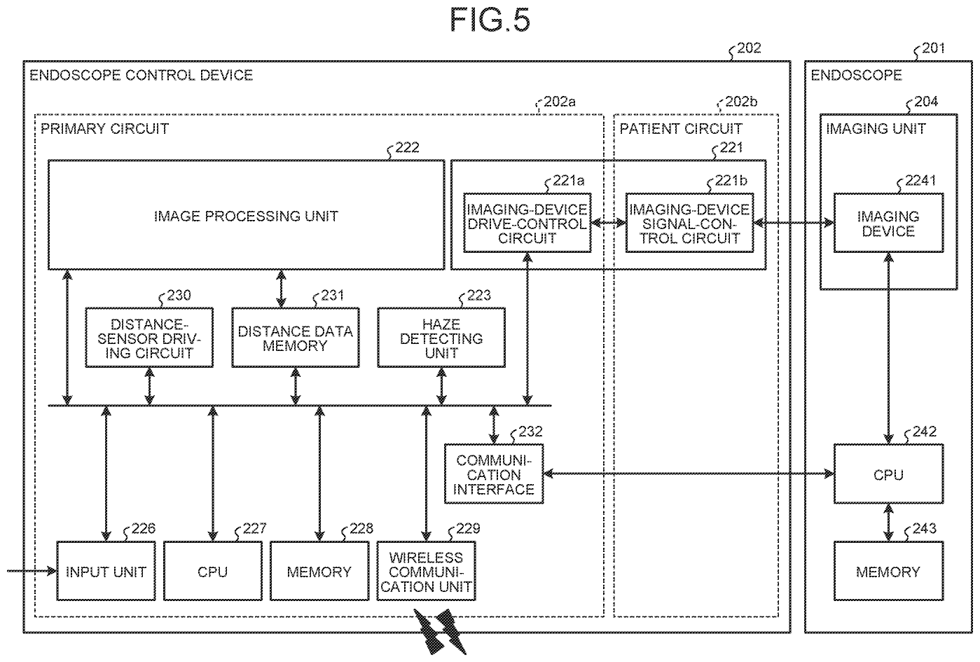

FIG. 5 is a block diagram illustrating a detailed functional configuration of the endoscope device 2.

As illustrated in FIG. 4 and FIG. 5, the endoscope device 2 includes an endoscope control device 202, a display device 203, an imaging unit 204 provided in the endoscope 201, and an operation input unit 205.

The endoscope control device 202 includes an imaging processing unit 221, an image processing unit 222, a haze detecting unit 223, an input unit 226, a CPU 227, a memory 228, a wireless communication unit 229, a distance-sensor driving circuit 230, a distance data memory 231, and a communication interface 232.

The imaging processing unit 221 includes an imaging-device drive-control circuit 221a that controls driving of an imaging device 2241 included in the imaging unit 204 provided in the endoscope 201, and an imaging-device signal-control circuit 221b that performs signal control of the imaging device 224a provided in a patient circuit 202b that is electrically insulated from a primary circuit 202a. The imaging-device drive-control circuit 221a is arranged in the primary circuit 202a. Moreover, the imaging-device signal-control circuit 221b is arranged in the patient circuit 202b that is electrically insulated from the primary circuit 202a.

The image processing unit 222 performs predetermined image processing with respect to input image data (PAW data), to output to the display device 203 through a bus. The image processing unit 222 is constituted of, for example, a processor having hardware, such as a digital signal processor (DSP) or a field programmable gate array (FPGA). The image processing unit 222 loads a program stored in the memory 228 to a work area of a memory to execute, and implements a functional module that meets a predetermined purpose by causing hardware and software to cooperate by controlling the respective components and the like through execution of the program by the processor. A detailed functional configuration of the image processing unit 222 will be described later.

The haze detecting unit 223 detects a haze in the field of view of the endoscope 201 inside the joint cavity Cl based on information relating to haze in the field of view of the endoscope 201. The information relating to haze includes, for example, a value acquired from image data generated by the endoscope 201, physical properties (turbidity) of the irrigation fluid, an impedance acquired from the treatment device 3, and the like.

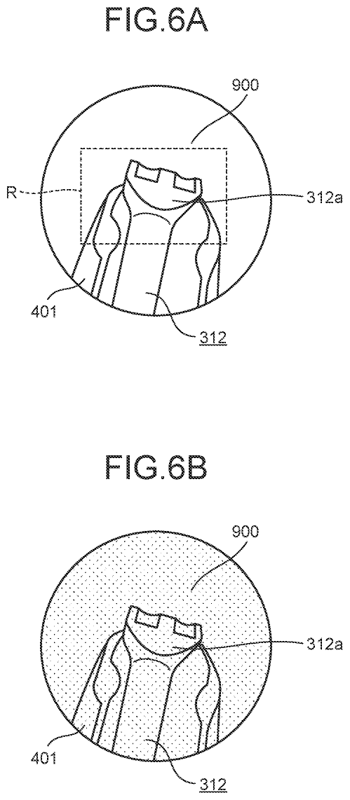

FIG. 6A is a diagram illustrating a state in which the field of view of the endoscope 201 is in a good condition. FIG. 6B is a diagram illustrating a state in which the field of view of the endoscope 201 is in a poor condition. Each of FIG. 6A and FIG. 6B schematically illustrates a display image corresponding to image data, which is the field of view of the endoscope 201, when a bone hole is formed in a femoral condyle 900 by an operator. Out of these, FIG. 6B is a schematic illustration of a state in which the field of view of the endoscope 201 is cloudy due to bone crushed into minute particles by driving of the ultrasound probe 312. In FIG. 6B, the minute bone is expressed by a dot.

As illustrated in FIG. 5, the input unit 226 accepts input of a signal input by the operation input unit 205 and input of signals from the respective devices constituting the treatment system 1.

The CPU 227 oversees and controls the operation of the endoscope control device 202. The CPU 227 loads a program stored in the memory 228 to a work area of a memory to execute, and controls operation of the respective components of the endoscope control device 202 by causing hardware and software to cooperate by controlling the respective components and the like through execution of the program by the processor.

The memory 228 stores various kinds of information necessary for the operation of the endoscope control device 202, various kinds of programs to be executed by the endoscope control device 202, image data acquired by the imaging unit 204, and the like. The memory 228 is constituted of, for example, a random access memory (RAM), a read only memory (ROM), a frame memory, and the like.

The wireless communication unit 229 is an interface to perform wireless communication with other devices. The wireless communication unit 229 is constituted of, for example, a communication module supporting Wi-Fi (registered trademark), Bluetooth (registered trademark), or the like.

The distance-sensor driving circuit 230 drives a not illustrated distance sensor that measures a distance to a predetermined object in an image captured by the imaging unit 204. In the first embodiment, the distance sensor may be arranged in the imaging device 2241. In this case, the imaging device 2241 may implement phase difference pixels capable of measuring a distance to the predetermined object from the imaging device 2241 in place of effective pixels. A time of flight (ToF) sensor may, of course, be provided near the distal end of the endoscope 201.

The distance data memory 231 stores distance data detected by the distance sensor. The distance data memory 231 is constituted of, for example, a RAM, a ROM, and the like.

The communication interface 232 is an interface to perform communication with the imaging unit 204.

The components described above except the imaging-device signal-control circuit 221b are arranged in the primary circuit 202a, and are connected to one another through a bus wiring.

The imaging unit 204 is arranged in the endoscope 201. The imaging unit 204 includes a imaging device 2241, a CPU 242, and a memory 243.

The imaging device 2241 generates image data by imaging a subject image that is formed by one or more optical systems not illustrated, and outputs the generated image data to the endoscope control device 202 under the control of the CPU 242. The imaging device 2241 is constituted of an imaging sensor, such as a charge coupled device (CCD) or a complementary metal oxide semiconductor (CMOS).

The CPU 242 oversees and controls the operation of the imaging unit 204. The CPU 242 loads a program stored in the memory 243 to a work area of a memory to execute, and controls the operation of the imaging unit 204 by causing hardware and software to cooperate by controlling the respective components and the like through execution of the program by the processor.

The memory 243 stores various kinds of information necessary for the operation of the imaging unit 204, various kinds of programs to be executed by the endoscope 201, image data generated by the imaging unit 204, and the like. The memory 243 is constituted of a RAM, a ROM, a frame memory, and the like.

The operation input unit 205 is composed of an input interface, such as a mouse, a keyboard, a touch panel, and a microphone, and accepts operation input of the endoscope device 2 by an operator.

### Functional Configuration of Treatment Device

Next, a functional configuration of the treatment device 3 will be explained.

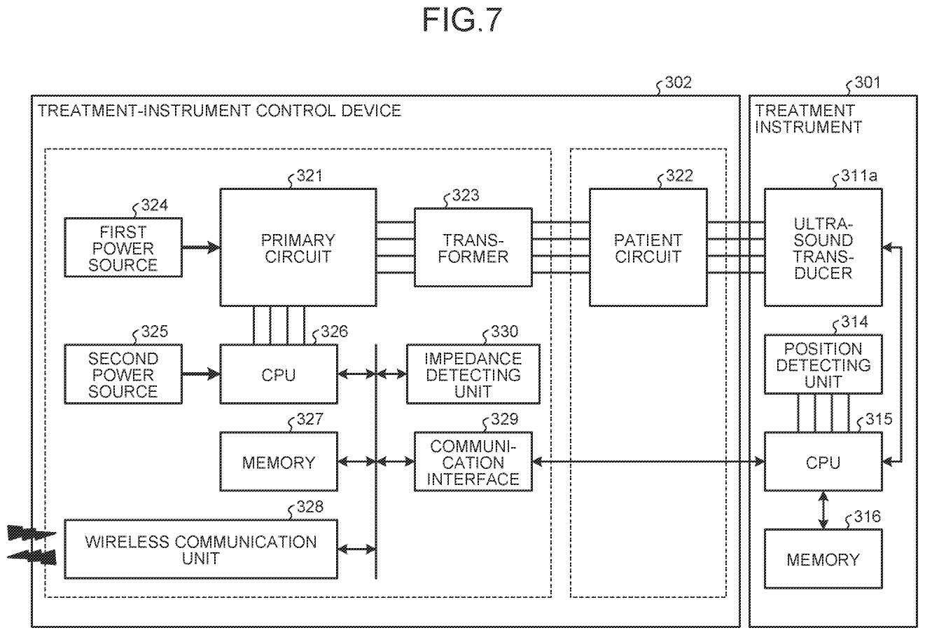

FIG. 7 is a block diagram illustrating a detailed functional configuration of the treatment device 3.

As illustrated in FIG. 4 and FIG. 7, the treatment device 3 includes a treatment instrument 301, a treatment-instrument control device 302, and an input/output unit 304.

The treatment instrument 301 includes an ultrasound transducer 312a, a position detecting unit 314, a CPU 315, and a memory 316.

The position detecting unit 314 detects a position of the treatment instrument 301, and outputs this detection result to the CPU 315. The position detecting unit 314 is constituted of at least one of an acceleration sensor and angular velocity sensor.

The CPU 315 oversees and controls the treatment instrument 301 including the ultrasound transducer 312a. The CPU 315 loads a program stored in the memory 316 to a work area of a memory to execute, and implements a functional module that meets a predetermined purpose by causing hardware and software to cooperate by controlling the respective components and the like through execution of the program by the processor.

The memory 316 stores various kinds of information necessary for the operation of the treatment instrument 301, various kinds of programs to be executed by the treatment instrument 301, identification information for identifying a type, a manufacturing date, performance, and the like of the treatment instrument 301.

The treatment-instrument control device 302 includes a primary circuit 321, a patient circuit 322, a transformer 323, a first power source 324, a second power source 325, a CPU 326, a memory 327, a wireless communication unit 328, a communication interface 329, and an impedance detecting unit 330.

The primary circuit 321 generates supply power to the treatment instrument 301. The patient circuit 322 is electrically insulated from the primary circuit 321. The transformer 323 electromagnetically connects the primary circuit 321 and the patient circuit 322. The first power source 324 is a high voltage power source that supplies driving power to the treatment instrument 301.

The second power source 325 is a low voltage power source that supplies driving power of a control circuit in the treatment-instrument control device 302.

The CPU 326 oversees and controls the operation of the treatment-instrument control device 302. The CPU 326 loads a program stored in the memory 327 to a work area of a memory to execute, and controls operation of the respective components of the treatment-instrument control device 302 by causing hardware and software to cooperate by controlling the respective components and the like through execution of the program by the processor.

The memory 327 stores various kinds of information necessary for the operation of the treatment-instrument control device 302, various kinds of programs to be executed by the treatment-instrument control device 302, and the like. The memory 327 is constituted of a RAM, a ROM, and the like.

The wireless communication unit 328 is an interface to perform wireless communication with other devices. The wireless communication unit 328 is constituted of, for example, a communication module supporting Wi-Fi (registered trademark), Bluetooth (registered trademark), or the like.

The communication interface 329 is an interface to perform communication with the treatment instrument 301.

The impedance detecting unit 330 detects an impedance at the time of driving of the treatment instrument 301, and outputs this detection result to the CPU 326. Specifically, the impedance detecting unit 330 is electrically connected, for example, between the first power source 324 and the primary circuit 321, detects an impedance of the treatment instrument 301 based on a frequency of the first power source 324, and outputs this detection result to the CPU 326.

The input/output unit 304 is constituted of an input interface, such as a mouse, a keyboard, a touch panel, and a microphone, and an output interface, such as a monitor and a speaker, and outputs performs operation input of the endoscope device 2 by an operator, and output of various kinds of information to notify to the operator.

### Functional Configuration of Irrigation Device

Next, a functional configuration of the irrigation device 5 will be explained.

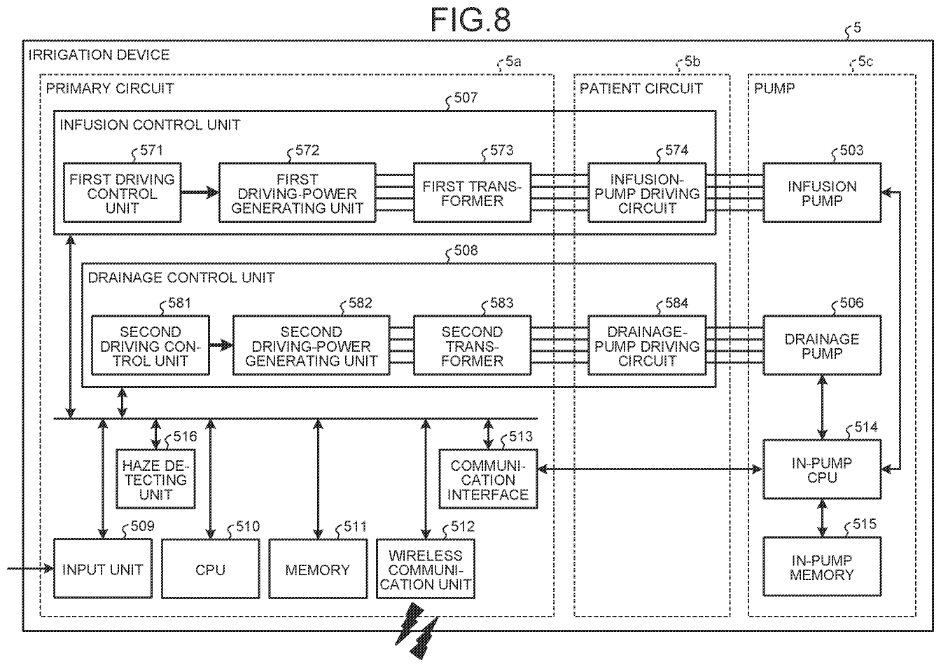

FIG. 8 is a block diagram illustrating a detailed functional configuration of the irrigation device 5.

As illustrated in FIG. 4 and FIG. 8, the irrigation device 5 includes the infusion pump 503, the drainage pump 506, an infusion control unit 507, a drainage control unit 508, an input unit 509, a CPU 510, a memory 511, a wireless communication unit 512, a communication interface 513, an in-pump CPU 514, an in-pump memory 515, and a haze detecting unit 516.

The infusion control unit 507 includes a first driving control unit 571, a first driving-power generating unit 572, a first transformer 573, and an infusion-pump driving circuit 574.

The first driving control unit 571 controls driving of the first driving-power generating unit 572 and the infusion-pump driving circuit 574.

The first driving-power generating unit 572 generates driving power of the infusion pump 503, and supplies this driving power to the first transformer 573.

The first transformer 573 electromagnetically connects the first driving-power generating unit 572 and the infusion-pump driving circuit 574.

In the infusion control unit 507 thus configured, the first driving control unit 571, the first driving-power generating unit 572, and the first transformer 573 are arranged in the primary circuit 5a. Moreover, the infusion-pump driving circuit 574 is arranged in a patient circuit 5b that is electrically insulated from the primary circuit 5a.

The drainage control unit 508 includes a second driving control unit 581, a second driving-power generating unit 582, a second transformer 583, and a drainage-pump driving circuit 584.

The second driving control unit 581 controls driving of the second driving-power generating unit 582 and the drainage-pump driving circuit 584.

The second driving-power generating unit 582 generates driving power of the drainage pump 506, and supplies the generated driving power to the second transformer 583.

The second transformer 583 electromagnetically connects the second driving-power generating unit 582 and the drainage-pump driving circuit 584.

In the drainage control unit 508 thus configured, the second driving control unit 581, the second driving-power generating unit 582, and the second transformer 583 are arranged in the primary circuit 5a. Moreover, the drainage-pump driving circuit 584 is arranged in the patient circuit 5b that is electrically insulated from the primary circuit 5a.

The input unit 509 accepts operation input not illustrated, or input of signals from the respective devices constituting the treatment system 1, and outputs the accepted signal to the CPU 510 and the in-pump CPU 514.

The CPU 510 and the in-pump CPU 514 oversees and controls the irrigation device 5 in collaboration. The CPU 510 loads a program stored in the memory 511 to execute, and controls operation of the respective components of the irrigation device 5 by causing hardware and software to cooperate by controlling the respective components and the like through execution of the program by the processor.

The memory 511 stores various kinds of information necessary for the operation of the irrigation device 5 and various kinds of programs executed by the irrigation device 5. The memory 511 is constituted of a RAM, a ROM, and the like.

The wireless communication unit 512 is an interface to perform wireless communication with other devices. The wireless communication unit 512 is constituted of, for example, a communication module supporting Wi-Fi, Bluetooth, or the like.

The communication interface 513 is an interface to perform communication with the infusion pump 503 and the endoscope 201.

The in-pump memory 515 stores various kinds of information necessary for the operation of the infusion pump 503 and the drainage pump 506, and various kinds of programs executed by the infusion pump 503 and the drainage pump 506.

The haze detecting unit 516 detects turbidity of the irrigation fluid based on at least one of physical properties, an absorbance, an impedance, and a resistance value of the irrigation fluid flowing inside the drainage tube 505, and outputs this detection result to the CPU 510.

In the irrigation device 5 thus configured, the input unit 509, the CPU 510, the memory 511, the wireless communication unit 512, the communication interface 513, and the haze detecting unit 516 are arranged in the primary circuit 5a. Furthermore, the in-pump CPU 514 and the in-pump memory 515 are arranged in a pump Sc. The in-pump CPU 514 and the in-pump memory 515 may be arranged near the infusion pump 503, or may be arranged near the drainage pump 506.

### Functional Configuration of Illumination Device

Next, a functional configuration of the illumination device 6 will be explained.

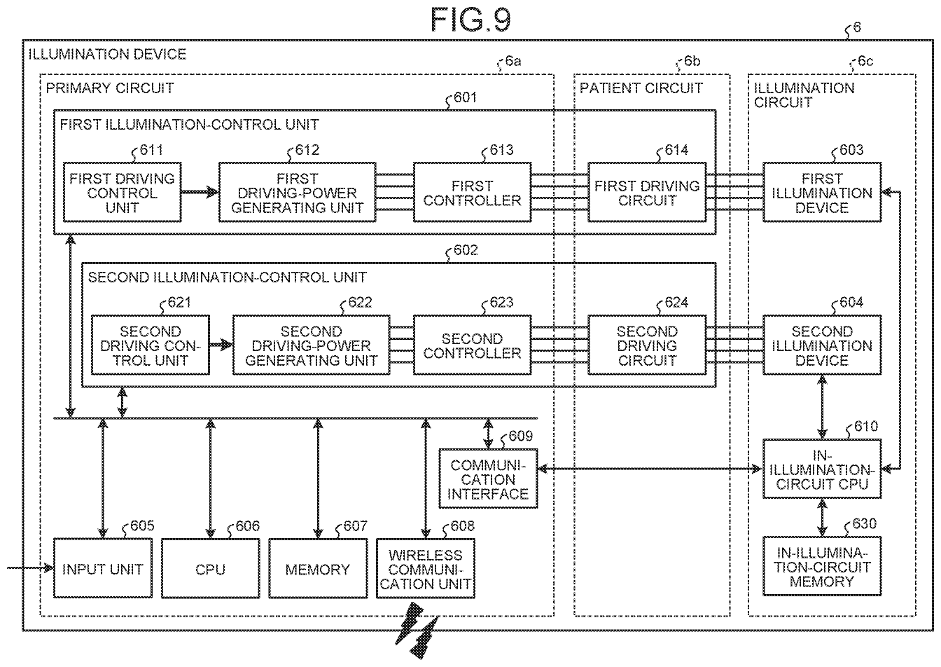

FIG. 9 is a block diagram illustrating a detailed functional configuration of the illumination device 6.

As illustrated in FIG. 4 and FIG. 9, the illumination device 6 includes a first illumination-control unit 601, a second illumination-control unit 602, a first illumination device 603, a second illumination device 604, an input unit 605, a CPU 606, a memory 607, a wireless communication unit 608, a communication interface 609, an in-illumination-circuit CPU 610, and an in-illumination-circuit memory 630.

The first illumination-control unit 601 includes a first driving control unit 611, a first driving-power generating unit 612, a first controller 613, and a first driving circuit 614.

The first driving control unit 611 controls driving of the first driving-power generating unit 612, the first controller 613, and the first driving circuit 614.

The first driving-power generating unit 612 generates driving power of the first illumination device 603, and outputs this driving power to the first controller 613 under control of the first driving control unit 611.

The first controller 613 controls light output of the first illumination device 603 by controlling the first driving circuit 614 according to the driving power input from the first driving-power generating unit 612.

The first driving circuit 614 drives the first illumination device 603 under the control of the first controller 613.

In the illumination-control unit 601 thus configured, the first driving control unit 611, the first driving-power generating unit 612, and the first controller 613 are arranged in a primary circuit 6a. Moreover, the first driving circuit 614 is arranged in a patient circuit 6b that is electrically insulated from the primary circuit 6a.

The second illumination-control unit 602 includes a second driving control unit 621, a second driving-power generating unit 622, a second controller 623, and a second driving circuit 624.

The second driving control unit 621 controls the second driving-power generating unit 622, the second controller 623, and the second driving circuit 624.

The second driving-power generating unit 622 generates driving power of the second illumination device 604, and outputs this driving power to the second controller 623 under the control of the second driving control unit 621.

The second controller 623 controls light output of the second illumination device 6 by controlling the second driving circuit 624 according to the driving power input from the second driving-power generating unit 622.

The second driving circuit 624 drives the second illumination device 604, and outputs illumination light under the control of the second controller 623.

In the second illumination-control unit 602 thus configured, the second driving control unit 621, the second driving-power generating unit 622, and the second controller 623 are arranged in the primary circuit 6a. Moreover, the second driving circuit 624 is arranged in the patient circuit 6b that is electrically insulated from the primary circuit 6a.

The first illumination device 603 irradiates light in a wavelength range of visible light (hereinafter, simply “visible light”) to the subject as the first illumination light to illuminate the subject through the endoscope 201. The visible light is white light (wavelength band λ=380 nm to 780 nm). The first illumination device 603 is constituted of, for example, a white light emitting diode (LED), a halogen lamp, or the like.

The second illumination device 604 irradiates light in a wavelength range outside visible light (hereinafter, simply “invisible light”) to the subject as the second illumination light to illuminate the subject through the endoscope 201. The invisible light is infrared light (wavelength band λ=800 nm to 2500 nm). The second illumination device 604 is constituted of, for example, an infrared LED lamp, or the like.

The input unit 605 accepts input of a signal from the respective devices constituting the treatment system 1, and outputs the accepted signal to the CPU 606 and the in-illumination-circuit CPU 610.

The CPU 606 and the in-illumination-circuit CPU 610 oversee and control the operation of the illumination device 6 in corporation. The CPU 606 loads a program stored in the memory 607 to a work area of a memory to execute, and controls operation of the respective components of the illumination device 6 by causing hardware and software to cooperate by controlling the respective components and the like through execution of the program by the processor.

The memory 607 stores various kinds of information necessary for the operation of the illumination device 6, and various kinds of programs to be executed by the illumination device 6. The memory 607 is constituted of, for example, a RAM, a ROM, and the like.

The wireless communication unit 608 is an interface to perform wireless communication with other devices. The wireless communication unit 608 is constituted of, for example, a communication module supporting Wi-Fi, Bluetooth, or the like.

The communication interface 609 is an interface to perform communication with an illumination circuit 6c.

The in-illumination-circuit memory 630 stores various kinds of information and a program necessary for the operation of the first illumination device 603 and the second illumination device 604. The in-illumination-circuit memory 630 is constituted of a RAM, a ROM, and the like.

In the illumination device 6 thus configured, the input unit 605, the CPU 606, the memory 607, the wireless communication unit 608, and the communication interface 609 are arranged in the primary circuit 6a. Furthermore, the first illumination device 603, the second illumination device 604, in-illumination-circuit CPU 610, and in-illumination-circuit memory 61A are arranged in the illumination circuit 6c.

### Configuration of Imaging Device

Next, a configuration of the imaging device 2241 described above will be explained.

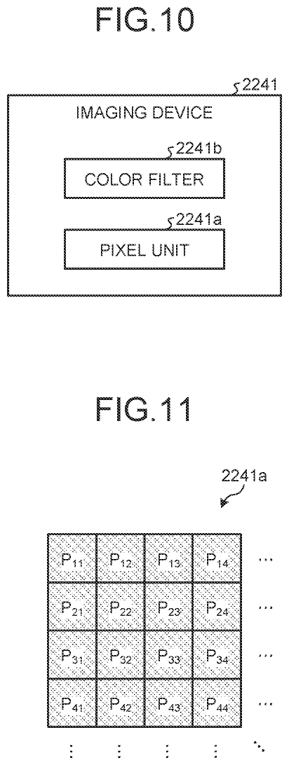

FIG. 10 is a block diagram illustrating a functional configuration of the imaging device 2241.

The imaging device 2241 illustrated in FIG. 10 is implemented by an imaging sensor of a CCD or a CMOS having multiple pixels arranged in two-dimensional matrix configuration. The imaging device 2241 performs photoelectric conversion with respect to a subject image (light) formed by the optical system not illustrated to generate image data (PAW data), and outputs this image data to the endoscope control device 202 under control of the CPU 242. The imaging device 2241 includes a pixel unit 2241a and a color filter 2241b.

First, a configuration of the pixel unit 2241a will be explained.

FIG. 11 is a diagram schematically illustrating a configuration of the pixel unit 2241a.

As illustrated in FIG. 11, the pixel unit 2241a is constituted of multiple pixels Pₙₘ (n=positive integer equal to or larger than 1, m=positive integer equal to or larger than 1), such as photodiodes that accumulates electric charges according to light intensity arranged in a two-dimensional matrix configuration. The pixel unit 2241a reads out an image signal from a pixel Pₙₘ of a readout region arbitrarily set as a readout target out of the multiple pixels Pₐm under control of the CPU 242, to output to the endoscope control device 202.

Next, a configuration of the color filter 2241b will be explained.

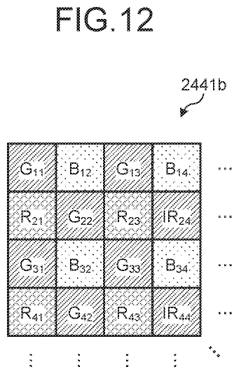

FIG. 12 is a diagram schematically illustrating a configuration of the color filter 2241b.

As illustrated in FIG. 12, the color filter 2241b includes a basic unit of a Bayer pattern element (RGGB) constituted of a filter R that passes light of a red wavelength band, two filters G that passes light of a green wavelength band, and a filter B that passes light of a blue wavelength band, and an IR unit (RGBIR) configured such that one of the filters G in the Bayer pattern is replaced with a filter IR that passes light of an infrared wavelength band.

In the color filter 2241b thus constituted, the basic units and the IR units are arranged at a predetermined interval. Specifically, in the color filter 2241b, the basic unit and the IR unit are arranged alternately with respect to the pixel unit 2241a.

The configuration of the color filter 2241b is not limited to the arrangement in which the basic unit and the IR unit are alternate and, for example, it may have a configuration in which one IR unit is arranged with respect to three basic units (interval of 3:1), and may be changed appropriately.

### Sensitivity Characteristics of Each Filter

Next, sensitivity characteristics of each filter will be explained.

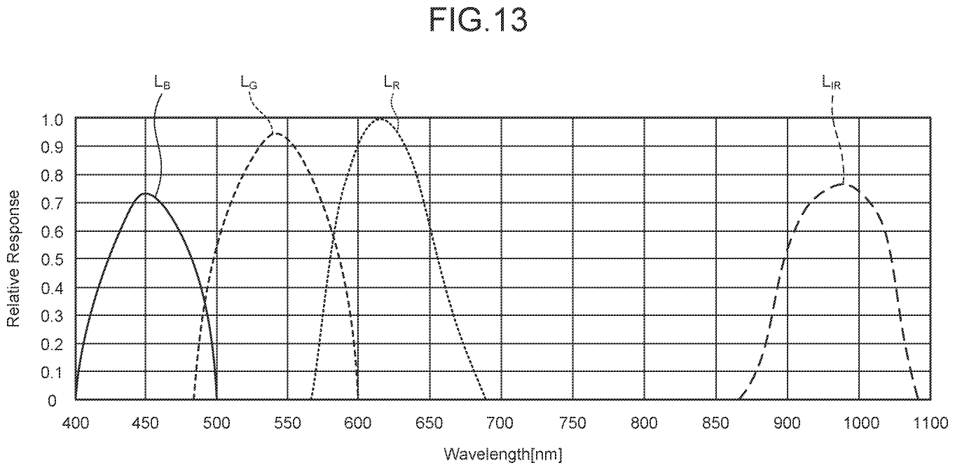

FIG. 13 is a diagram schematically illustrating a sensitivity and wavelength band of each filter.

In FIG. 13, a horizontal axis represents wavelength (nm) and a vertical axis represents transmittance characteristic (sensitivity characteristic). Moreover, in FIG. 13, a curve L_{B} represents the transmittance characteristic of the filter B, a curve L_{G} represents the transmittance characteristic of the filter G, a curve L_{R} represents the transmittance characteristic of the filter R, and a curve L_{IR} represents the transmittance characteristic of the filter IR.

As indicated by the curve L_{B} in FIG. 13, the filter B passes light of a blue wavelength band (400 nm to 500 nm). Moreover, as indicated by the curve L_{G} in FIG. 13, the filter G passes light of a green wavelength band (480 nm to 600 nm). Furthermore, as indicated by the curve L_{R} in FIG. 13, the filter R passes light of a red wavelength band (570 nm to 680 nm). Moreover, as indicated by the curve L_{IR} in FIG. 13, the filter IR passes light of an infrared wavelength band (870 nm to 1080 nm). In the following, it will be explained by representing a pixel Pₙₘ with the red filter R placed on a light receiving surface as R pixel, the pixel Pₙₘ with the green filter G placed on the light receiving surface as G pixel, the pixel Pₙₘ with the blue filter B placed on the light receiving surface as B pixel, and the pixel Pₙₘ with the infrared filter IR placed on the light receiving surface as IR pixel.

### Detailed Functional Configuration of Image Processing Unit

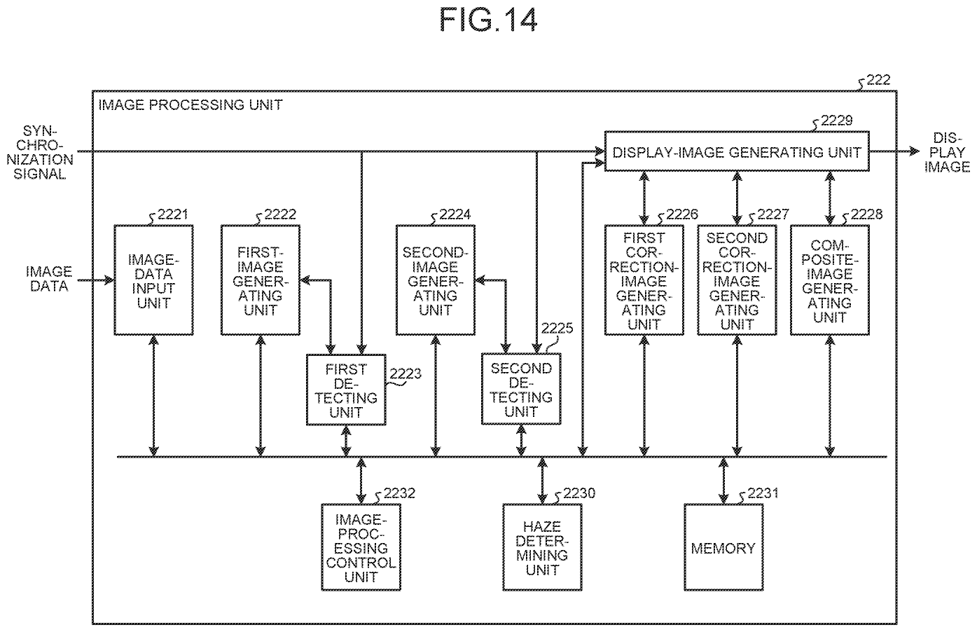

Next, a detailed functional configuration of the image processing unit 222 described above will be explained. FIG. 14 is a block diagram illustrating a detailed functional configuration of the image processing unit 222. The image processing unit 222 illustrated in FIG. 14 includes an image-data input unit 2221, a first-image generating unit 2222, a first detecting unit 2223, a second-image generating unit 2224, a second detecting unit 2225, a first correction-image generating unit 2226, a second correction-image generating unit 2227, a composite-image generating unit 2228, a display-image generating unit 2229, a haze determining unit 2230, a memory 2231, and an image-processing control unit 2232.

The image-data input unit 2221 accepts input of image data generated by the endoscope 201 and input of a signal from the respective devices constituting the treatment system 1, and outputs the accepted data and the signal to the bus.

The first-image generating unit 2222 performs predetermined image processing with respect to the image data (PAW data) input through the image-data input unit 2221 to generate first image data in accordance with a synchronization signal synchronized with imaging drive of the imaging unit 204, and outputs this first image data to the first detecting unit 2223, the first correction-image generating unit 2226, and the composite-image generating unit 2228. Specifically, the first-image generating unit 2222 generates the first image data (normal color image data) based on pixel values of the R pixel, the G pixel, and the B pixel included in the image data. The predetermined image processing includes, for example, demosaicing processing, color correction processing, black-level correction processing, noise reduction processing, and y correction processing, and the like. In this case, the first-image generating unit 2222 generates the first image data by interpolating the pixel value of the IR pixel using the pixel values of surrounding pixels, for example, an adjacent G pixel. The first-image generating unit 2222 may perform the demosaicing processing by interpolating the pixel value of the IR pixel, pixel-defect correction processing of color image data, and the like by using other publicly-known technique. In the first embodiment, the first-image generating unit 2222 functions as a first-image acquiring unit that acquires a first image including a region in which a living body is treated with an energy treatment instrument, such as the ultrasound probe 312. The first-image generating unit 2222 may generate the first image data based on a driving signal of the treatment instrument 301 also.

The first detecting unit 2223 detects changes in tone from at least a part of the first image corresponding to the first image data (hereinafter, simply “first image”) based on the first image data generated by the first-image generating unit 2222, and outputs this detection result to the first correction-image generating unit 2226, the composite-image generating unit 2228, and the image-processing control unit 2232. Specifically, the first detecting unit 2223 detects haze in the field of view of the endoscope 201 as a part of the region in the first image based on the first image generated by the first-image generating unit 2222, and outputs this detection result to the first correction-image generating unit 2226, the composite-image generating unit 2228, and the image-processing control unit 2232. As for the detecting method of haze by the first detecting unit 2223, detailed explanation of the detecting method is omitted because haze components of a haze estimating unit 2226a of the first correction-image generating unit 2226 described later are detected by a similar method.

The haze of the field of view of the endoscope 201 is a degree of turbidity caused by bone powder or debris dissolved in the irrigation fluid that is a cause of deterioration in tones in the first image. As the cause of deterioration of the image quality, in addition to a phenomenon caused by dissolution of living tissues, such as bone powder, debris, blood, and bone marrow, into the irrigation fluid, a phenomenon of smoke and sparks during a procedure by the treatment instrument 301 is also considered. In the following, a state in which the irrigation fluid has become turbid because of dissolution of bone powder into the irrigation fluid will be explained. Because the irrigation fluid in which a living tissue has dissolved becomes turbid and opaque in white overall, it has characteristics of having high brightness, low saturation (poor color reproduction), and low contrast. Therefore, the first detecting unit 2223 detects the haze (haze components) of the field of view of the endoscope 201 by calculating contrast, brightness, and saturation as the haze of the field of view of the endoscope 201 for each pixel constituting the first image.

The second-image generating unit 2224 performs predetermined image processing with respect to image data (RAW data) input through the image-data input unit 2221 in accordance with the synchronization signal that is synchronized with imaging drive of the imaging unit 204 to generate the second image data, and outputs this second image data to the second detecting unit 2225, the second correction-image generating unit 2227, and the composite-image generating unit 2228. Specifically, the second-image generating unit 2224 generates the second image data (infrared image data) based on pixel values of the IR pixels included in the image data. The predetermined image processing includes, for example, demosaicing processing, color correction processing, block-level correction processing, noise reduction processing, y correction processing, and the like. In this case, the second-image generating unit 2224 generates the second image data by using a pixel value of an IR pixel in a target pixel and a pixel value of a surrounding IR pixel around the IR pixel in the target pixel for interpolation.

The second-image generating unit 2224 may perform interpolation of pixel values of the IR pixel by using another publicly-known technique. In the first embodiment, the second-image generating unit 2224 functions as a second-image acquiring unit that acquires the second image data that differs in wavelength from the first image. The second-image generating unit 2224 may generate the second image data based on a driving signal of the treatment instrument 301 also.

The second detecting unit 2225 detects an edge component from at least a part of a second image corresponding to the second image data (hereinafter, simply “second image”) based on the second image data generated by the second-image generating unit 2224, and outputs this detection result to the second correction-image generating unit 2227, the composite-image generating unit 2228, and the image-processing control unit 2232. Specifically, the second detecting unit 2225 detects and edge component of a region including the endoscope 201 as at least a part of the region of the second image based on the second image (infrared image) generated by the second-image generating unit 2224, and outputs this detection result to the second correction-image generating unit 2227, the composite-image generating unit 2228, and the image-processing control unit 2232. The second detecting unit 2225 detects an edge component from the second image, for example, by a publicly-known edge extraction processing. Moreover, the second detecting unit 2225 may detect a change in tone from at least a part of the region of the second image by a similar method to that of the first detecting unit 2223.

The first correction-image generating unit 2226 generates first correction-image data by performing tone correction with respect to the first image input from the first-image generating unit 2222 based on the detection result input from the first detecting unit 2223 in accordance with the synchronization signal synchronized with imaging drive of the imaging unit 204, and outputs a first correction image corresponding to this first correction-image data (hereinafter, simply, “first correction image”) to the composite-image generating unit 2228 or the display-image generating unit 2229. Specifically, the first correction-image generating unit 2226 generates the first correction image from which a visibility deterioration factor due to haze (haze components) included in the first image is removed, and outputs this first correction image to the composite-image generating unit 2228 or the display-image generating unit 2229. Details of the first correction-image generating unit 2226 will be described later.

The second correction-image generating unit 2227 generates second correction-image data by performing tone correction with respect to the second image input from the second-image generating unit 2224 based on the detection result input from the second detecting unit 2225 in accordance with the synchronization signal synchronized with imaging drive of the imaging unit 204, and outputs this second correction-image data (hereinafter, simply, “second correction image”) to the composite-image generating unit 2228 or the display-image generating unit 2229. Specifically, the second correction-image generating unit 2227 performs edge extraction processing to extract an edge component that deteriorates the visibility with haze (haze component) with respect to the second image, and generates the second correction image obtained by performing edge enhancement processing to enhance an edge with respect to this extracted edge component.

The composite-image generating unit 2228 generates composite image data by combining the first correction image input from the first correction-image generating unit 2226 and the second correction image input from the second correction-image generating unit 2227 at a predetermined ratio under control of the image-processing control unit 2232, and outputs a composited image corresponding to this composite image data (hereinafter, simply “composite image”) to the display-image generating unit 2229. The predetermine ratio is, for example, 5:5. The composite-image generating unit 2228 may change the ratio of combining the first correction image and the second correction image based on the ratio of the respective detection results of the first detecting unit 2223 and the second detecting unit 2225 appropriately, and may change the combining ratio of combining the first correction image and the second correction image according to a component and a kind of the haze. The composite-image generating unit 2228 may generate a composite image by adding an edge component extracted from the second correction image by the second detecting unit 2225 to the first correction image.

The display-image generating unit 2229 generates a display image corresponding to display data to be displayed on the display device 203 based on at least one of the first image input from the first-image generating unit 2222, the second image input from the second-image generating unit 2224, the first correction image input from the first correction-image generating unit 2226, the second correction image input from the second correction-image generating unit 2227, and the composite image input from the composite-image generating unit 2228 according to the synchronization signal that is synchronized with imaging drive of the imaging unit 204 under control of the image-processing control unit 2232, and outputs it to the display device 203. Specifically, the display-image generating unit 2229 converts the format of an input image into a predetermined format, for example, converting from the RGB format to the YCbCr format, to output to the display device 203. The display image generated by the display-image generating unit 2229 includes temporally continuous images in the field of view of the endoscope 201. The display-image generating unit 2229 may generate a display image based on a driving signal of the treatment instrument 301.

The haze determining unit 2230 determines whether the haze detected by the first detecting unit 2223 is equal to or larger than a predetermined value, and outputs this determination result to the image-processing control unit 2232. The predetermined value is, for example, a value corresponding to a level at which a treatment site in the field of view of the endoscope 201 becomes obscured by the haze. For example, as a value of the level at which a treatment site becomes obscured is a value of high brightness and low saturation (high-brightness white.

The memory 2231 stores various kinds of information necessary for the operation of the image processing unit 222, various kinds of programs executed by the image processing unit 222, various kinds of image data, and the like. The memory 2231 is constituted of a RAM, a ROM, a frame memory, and the like.

The image-processing control unit 2232 controls the respective components constituting the image processing unit 222. The image-processing control unit 2232 loads a program stored in the memory 2231 to a work area of a memory to execute, and controls operation of the respective components of the image processing unit 222 by causing hardware and software to cooperate by controlling the respective components and the like through execution of the program by the processor.

### Detailed Functional Configuration of First Correction-Image Generating Unit

Next, a detailed functional configuration of the first correction-image generating unit 2226 will be explained.

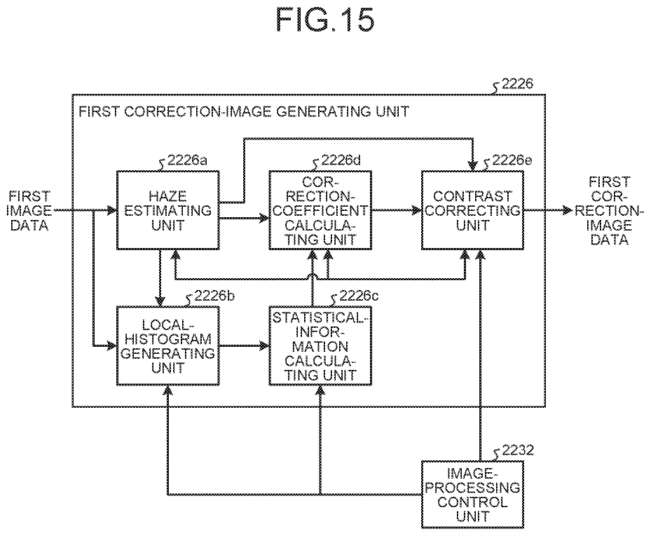

FIG. 15 is a block diagram illustrating a detailed functional configuration of the first correction-image generating unit 2226.

The first correction-image generating unit 2226 illustrated in FIG. 15 includes the haze estimating unit 2226a, a local-histogram generating unit 2226b, a statistical-information calculating unit 2226c, a correction-coefficient calculating unit 2226d, and a contrast correcting unit 2226e.

The haze estimating unit 2226a estimates a haze component of each pixel in the first image. The haze component of each pixel is a degree of turbidity caused by bone powder or debris dissolved in the irrigation fluid that is a cause of deterioration in tones in the first image. As the cause of deterioration of the image quality, in addition to a phenomenon caused by dissolution of living tissues, such as bone powder, debris, blood, and bone marrow, into the irrigation fluid, a phenomenon of smoke and sparks during a procedure by the treatment instrument 301 is also considered. In the following, a state in which the irrigation fluid has become turbid because of dissolution of bone powder into the irrigation fluid will be explained. Because the irrigation fluid in which a living tissue has dissolved has characteristics of having high brightness, low saturation (poor color reproduction), and low contrast.

Therefore, the haze estimating unit 2226a estimates a haze component in the field of view of the endoscope by calculating the contrast, or the brightness and saturation of the first image. Specifically, the haze estimating unit 2226a estimates a haze component H(x, y) based on an R value, a G value, and a B value of a pixel at coordinates (x, y) in the first image.

When the R value, the G value, and the B value at the coordinates (x, y) are Ir, Ig, and Ib, respectively, the haze component H(x, y) of the pixel at the coordinates (x, y) is estimated by following Equation (1).

$$H ( x , y ) = min ( Ir , Ig , Ib ) \qquad ( 1 )$$

The haze estimating unit 2226a performs calculation of Equation (1) described above for each pixel of the first image. The haze estimating unit 2226a sets a scan region F (small region) of a predetermined size with respect to the first image. The size of this scan region F is, for example, pixels of a predetermined size, m×n (m, n are positive integers). In the following explanation, the pixel at the center of the scan area F will be referred to as the reference pixel. Furthermore, in the following explanation, the respective pixels around the reference pixel in the scan region F will be referred to as neighboring pixels. Furthermore, in the following explanation, the scan region F will be explained as being formed with a size of, for example, 5×5 pixels. Of course, the scan region F is also applicable to a single pixel.

The haze estimating unit 2226a calculates (Ir, Ig, Ib) of each pixel in the scan region F while shifting the position of the scan region F with respect to the first image, and estimates the smallest values out of those as the haze component H(x, y) of the reference pixel. In the first image, the pixel values in the high-brightness and low-saturation region have the R value, the G value, and the B value that are equal and large and, therefore, values of min(Ir, Ig, Ib) become large. That is, the high-brightness and low-saturation region have large values for the haze component H(x, y).

On the other hand, the pixel values in a low-brightness or high-saturation region have at least one of the R value, the G value, and the B value that is low and, therefore, values of min(Ir, Ig, Ib) become small. That is, the low-brightness and high saturation region have small values for the haze component H(x, y).

As described, the haze component H(x, y) has a larger value as the concentration of bone powder dissolved in the irrigation fluid is higher (the whiteness of the bone powder is more significant) and a smaller value as the concentration of bone powder is lower. In other words, the haze component H(x, y) increases as the color (whiteness) of the irrigation fluid becomes more intense due to the bone powder dissolved in the irrigation fluid, and decreases as the color of the irrigation fluid becomes less intense.

The haze estimating unit 2226a estimates the haze component H(x, y) by using Equation (1) described above, but it is not limited thereto, and any indicator that indicates high brightness and low saturation can be used as the haze component. The haze estimating unit 2226a may use at least one of a local contrast value, an edge strength, a color density, and a subject distance to estimate the haze component. Moreover, the first detecting unit 2223 and the second detecting unit 2225 described above detect haze (haze component) by a method similar to that of the haze estimating unit 2226a.

The local-histogram generating unit 2226b determines a distribution of a histogram in a local region including the reference pixel of the first image and the neighboring pixels of this reference pixel based on the haze component H(x, y) input from the haze estimating unit 2226a. This degree of change in the haze component H(x, y) becomes an indicator for determining a region to which each pixel belongs in the local region. Specifically, this degree of change in the haze component H(x, y) is determined based on a difference in the haze component H(x, y) between the reference pixel and the neighboring pixels within the local region.

That is, the local-histogram generating unit 2226b generates a brightness histogram for the local region including the neighboring pixels for each reference pixel based on the first image input from the first-image generating unit 2222 and the haze component H(x, y) input from the haze estimating unit 2226a. Generating a typical histogram is performed by regarding pixel values in a target local region as brightness values, and by counting the frequency of each pixel value by one.

On the other hand, the local-histogram generating unit 2226b according to the first embodiment assigns weight to the count values for the pixel values of the neighboring pixels according to the haze component H(x, y) of the reference pixel and the neighboring pixels within the local region. The count value for the pixel values of the neighboring pixels take a value, for example, within a range of 0.0 to 1.0. Moreover, the count value is set to have a smaller value as the difference in the haze component H(x, y) between the reference pixel and the neighboring pixels increases, and is set to have a larger value as the difference in the haze component H(x, y) between the reference pixel and the neighboring pixels decreases. Furthermore, the local region is formed with, for example, a size of 7×7 pixels.

In generation of a typical histogram, if the histogram is created using only brightness, the brightness of a neighboring pixel having a significant difference from the brightness of the target pixel is also counted in the same way. The local histogram is preferable to be generated according to an image region to which the target pixel belongs.

On the other hand, in generation of the brightness histogram in the first embodiment, the count value for the pixel value of the respective pixels in the local region in the first image data is set according to the difference in the haze component H(x, y) between the reference pixel and the respective neighboring pixels within the local region in the first image data of the haze component H(x, y). Specifically, the count value is calculated, for example, by using a Gaussian function (for example, U.S. Pat. No. 6,720,012 or U.S. Pat. No. 6,559,229, note that the blurriness component is replaced with the haze component) such that the count value becomes smaller as the difference in the haze component H(x, y) between the reference pixel and the neighboring pixel increases, and becomes larger as the difference in the haze component H(x, y) between the reference pixel and the neighboring pixel decreases.

The method of calculating the count value by the local-histogram generating unit 2226b is not limited to the one using a Gaussian function as long as it is possible to set the count value to become smaller as the difference between the reference pixel and the neighboring pixel increases. For example, the local-histogram generating unit 2226b may calculate the count value using a lookup table or a line approximation table instead of a Gaussian function.

Moreover, the local-histogram generating unit 2226b may be configured to compare the difference in the value between the reference pixel and the neighboring pixel with a threshold, and to decrease the count value of the neighboring pixel (for example, to 0.0) when it is equal to or larger than the threshold.

Furthermore, the local-histogram generating unit 2226b is not necessarily required to use the frequency of pixel value as the count value. For example, the local-histogram generating unit 2226b may use each of the R value, the G value, and the B value as the count value. Moreover, the local-histogram generating unit 2226b may use the G value for the count value as the brightness value.

The statistical-information calculating unit 2226c calculate representative brightness based on statistical information of the brightness histogram that is input from the local-histogram generating unit 2226b. The representative brightness includes the brightness of a low brightness region, the brightness of a high brightness region, and the brightness of an intermediate brightness region within an effective brightness range of the brightness histogram. The brightness of the low brightness region is the minimum brightness in the effective brightness range. The brightness of the high brightness region is the maximum brightness in the effective brightness range. The brightness of the intermediate brightness region is mean brightness. The minimum brightness is the brightness at which the cumulative frequency is at 5% of the maximum value in a cumulative histogram generated from the brightness histogram. The maximum brightness is the brightness at which the cumulative frequency is at 95% of the maximum value in the cumulative histogram generated from the brightness histogram. The mean brightness is the brightness at which the cumulative frequency is at 50% of the maximum value in the cumulative histogram generated from the brightness histogram.

Furthermore, the percentages of the cumulative frequency corresponding to the minimum brightness, the maximum brightness, and the mean brightness, namely 5%, 50%, and 95%, respectively, can be changed appropriately. Moreover, while the brightness of the intermediate brightness region is defined as the mean brightness in the cumulative histogram, it is not limited thereto, and the mean brightness is not necessarily required to be calculated from the cumulative frequency. For example, the brightness corresponding to the maximum frequency in the brightness histogram can also be applied as the brightness in the intermediate brightness region.

The correction-coefficient calculating unit 2226d calculates a correction coefficient to correct the contrast in the local region based on the haze component H(x, y) input from the haze estimating unit 2226a and the statistical information input from the statistical-information calculating unit 2226c. Specifically, when the contrast correction is performed by histogram stretching, the correction-coefficient calculating unit 2226d calculates a coefficient for the histogram stretching by using the mean brightness and the maximum brightness out of the statistical information.

The histogram stretching is processing to enhance a contrast by expanding an effective brightness range of a histogram (for example, refer to U.S. Pat. No. 6,720,012 or U.S. Pat. No. 6,559,229). The correction-coefficient calculating unit 2226d uses histogram stretching as a method to achieve the contrast correction, but it is not limited thereto. As the method to achieve the contrast correction, for example histogram equalization may be used. For example, the correction-coefficient calculating unit 2226d may use a method, such as a method using a cumulative histogram or a line approximation table, as the method of achieving the histogram equalization. This cumulative histogram accumulates the frequency values of the brightness histogram sequentially.

The contrast correcting unit 2226e performs, with respect to the first image input from the first-image generating unit 2222, contrast correction of the reference pixel in the first image data based on the haze component H(x, y) input from the haze estimating unit 2226a and the correction coefficient input from the correction-coefficient calculating unit 2226d (for example, refer to U.S. Pat. No. 6,720,012 or U.S. Pat. No. 6,559,229).

The first correction-image generating unit 2226 thus configured estimates the haze component H(x, y) based on the first image, calculates the brightness histogram and the representative brightness using this estimation result, calculates the correction coefficient to correct the contrast in the local region, and performs the contrast correction based on the haze component H(x, y) and the correction coefficient. The first correction-image generating unit 2226 can thereby generate the first correction image obtained by removing the haze from the first image.

### Overview of Treatment

Next, an overview of a treatment performed by an operator by using the treatment system 1 will be explained.

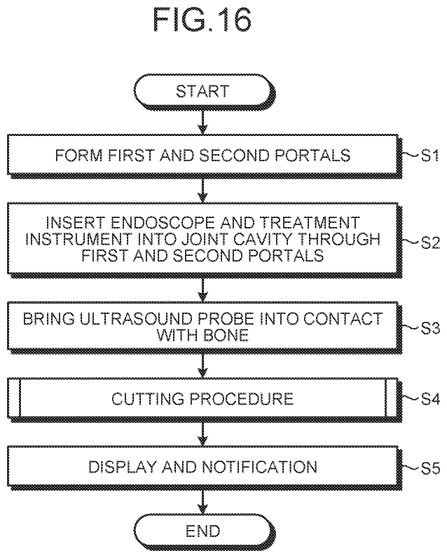

FIG. 16 is a flowchart explaining the overview of the treatment performed by an operator using the treatment system 1. The operator that performs the treatment may be a single surgeon or may be two or more operators including a surgeon and an assistant.

As illustrated in FIG. 16, the operator forms the first portal P1 and the second portal P2 that respectively connect the inside of the joint cavity Cl of the knee joint J1 and the outside of the skin (step S1).

Subsequently, the operator inserts the endoscope 201 into the joint cavity Cl through the first portal P1, inserts the guiding device 4 into the joint cavity Cl through the second portal P2, and inserts the treatment instrument 301 into the joint cavity Cl, guided by the guiding device 4 (step S2). Although a case in which the endoscope 201 and the treatment instrument 301 are inserted into the joint cavity Cl through the first portal P1 and the second portal P2 after forming the two portals has been explained herein, the second portal P2 may be formed to insert the guiding device 4 and the treatment instrument 301 into the joint cavity Cl after the first portal P1 is formed and the endoscope 201 is inserted into the joint cavity Cl.

Thereafter, the operator brings the ultrasound probe 312 into contact with a bone to be treated while confirming the endoscopic image within the joint cavity Cl displayed on the display device 203 (step S3).

Subsequently, the operator performs a cutting procedure using the treatment instrument 301 while viewing the endoscopic image displayed on the display device 203 (step S4). Details of the processing of the treatment system 1 in the cutting procedure will be described later.

Thereafter, the display device 203 performs display and notification processing for displaying the inside of the joint cavity Cl and information relating to a state after the cutting procedure (step S5). The endoscope control device 202 stops the display and notification, for example, after a predetermined time has passed since the display and notification processing is started. The operator ends the treatment using the treatment system 1.

### Details of Cutting Procedure

Next, details of the cutting procedure at step S4 in FIG. 16 described above will be explained.

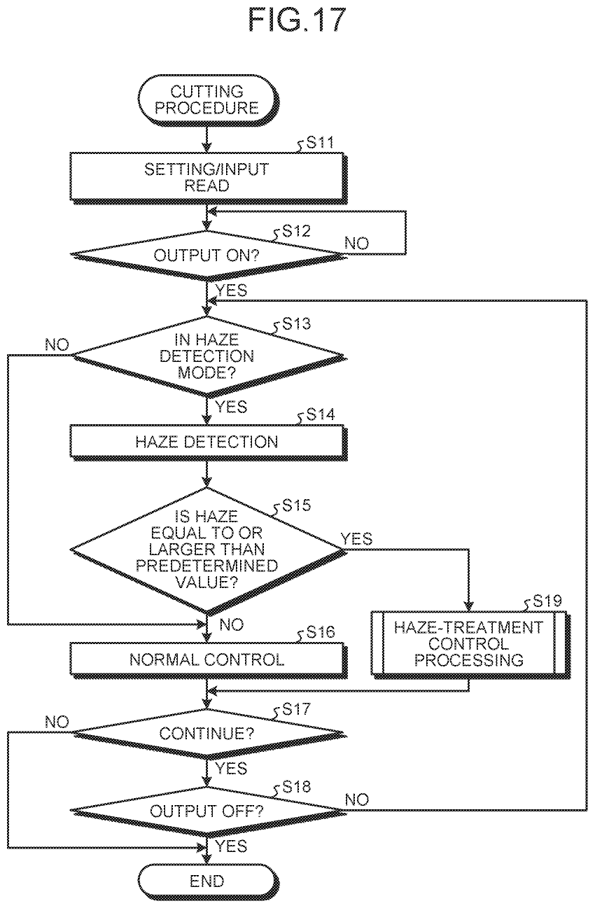

FIG. 17 explains an overview of the processing performed in the cutting procedure by the endoscope control device 202.

In the following, each processing is explained to be performed under control of CPUs of the respective control devices, but the processing may be performed collectively by either one of the control devices, such as the network control device 7, for example.

The CPU 227 performs communication with the respective devices, and performs setting of control parameters for each of the treatment device 3 and the irrigation device 5, and input of the control parameters for each of the treatment device 3 and the irrigation device 5 (step S11).

Subsequently, the CPU 227 determines whether the respective devices constituting the treatment system 1 have become an output ON state (step S12). When it is determined that the devices of the respective components constituting the treatment system 1 have become the output ON state by the CPU 227 (step S12: YES), the endoscope control device 202 shifts to step S13 described later. On the other hand, when it is determined that the devices of the respective components constituting the treatment system 1 have not become the output ON state by the CPU 227 (step S12: NO), the CPU 227 continues this determination until the devices of the respective components constituting the treatment system 1 become the output ON state.

At step S13, the CPU 227 determines whether the observation mode of the endoscope control device 202 in the treatment system 1 is set to the haze detection mode. When it is determined that the observation mode of the endoscope control device 202 in the treatment system 1 is set to the haze detection mode by the CPU 227 (step S13: YES), the endoscope control device 202 shifts to step S14 described later. On the other hand, when it is determined that the observation mode of the endoscope control device 202 in the treatment system 1 is not set to the haze detection mode (step S13: NO), the endoscope control device 202 shifts to step S16 described later.

At step S14, the haze detecting unit 223 detects haze in the field of view of the endoscope 201 based on either one of the first image generated by the endoscope, the detection result of the impedance detecting unit 330 of the treatment-instrument control device 302, and the detection result of the haze detecting unit 516 of the irrigation device 5. Specifically, the haze detecting unit 223 detects the haze in the field of view of the endoscope 201 by using either one of the brightness and the contrast of the first image when the first image generated by the endoscope is used. Moreover, the haze detecting unit 223 detects the haze in the field of view of the endoscope 201 based on a change rate of impedance when the impedance detected by the impedance detecting unit 330 of the treatment-instrument control device 302 is used. Furthermore, the haze detecting unit 223 detects the haze in the field of view of the endoscope 201 based on turbidity of the irrigation fluid detected by the haze detecting unit 516 of the irrigation device 5 when the detection result of the haze detecting unit 516 of the irrigation device 5 is used.

Subsequently, the CPU 227 determines whether the haze in the field of view of the endoscope 201 is equal to or larger than a predetermined value based on the detection result detected by the haze detecting unit 223 (step S15).

Specifically, the CPU 227 determines whether an average value of a sum of luminance of each pixel in the first image detected by the haze detecting unit 223 is equal to or larger than the predetermined value when the haze detecting unit 223 uses the first image. The predetermined value as the luminance is a high brightness value close to white. In this case, the CPU 227 determines that there is a haze in the field of view of endoscope 201 when the average value of the sum of the luminance of the respective pixels in the first image detected by the haze detecting unit 223 is equal to or larger than the predetermined value. On the other hand, the CPU 227 determines that there is no haze in the field of view of endoscope 201 when the average value of a sum of the brightness and the saturation of the respective pixels in the first image detected by the haze detecting unit 223 is not equal to or larger than the predetermined value.

Moreover, the CPU 227 determines whether the impedance is equal to or larger than a predetermined value when the haze detecting unit 223 uses the impedance detected by the impedance detecting unit 330. When the impedance detected by the impedance detecting unit 330 is equal to or larger than the predetermined value, the haze detecting unit 223 determines that there is a haze in the field of view of the endoscope 201. On the other hand, when the impedance detected by the impedance detecting unit 330 is not equal to or larger than the predetermined value, the haze detecting unit 223 determines that there is no haze in the field of view of the endoscope 201.

Furthermore, the CPU 227 determines whether the turbidity of the irrigation fluid is equal to or larger than a predetermined value when the haze detecting unit 223 uses the turbidity of the irrigation fluid detected by the haze detecting unit 516 of the irrigation device 5. When the turbidity of the irrigation fluid detected by the haze detecting unit 223 is equal to or larger than a predetermined value, the CPU 227 determines that there is a haze in the field of view of the endoscope 201. On the other hand, when the turbidity of the irrigation fluid detected by the haze detecting unit 223 is not equal to or larger than the predetermined value, it is determined that there is no haze in the field of view of the endoscope 201.

At step S15, when it is determined that there is a haze in the field of view of the endoscope 201 by the CPU 227 (at step S15: YES), the endoscope control device 202 shifts to step S19 described later. On the other hand, when it is determined that there is no haze in the field of view of the endoscope 201 (step S15: NO), the endoscope control device 202 shifts to step S16 described later.

At step S16, the CPU 227 performs normal control with respect to the endoscope control device 202. Specifically, the CPU 227 outputs the first image (color image) generated by the image processing unit 222 to the display device 203 to display. Thus, the operator can perform treatment by using the treatment instrument 301 while viewing the first image displayed on the display device 203 even when the visibility around the treatment site is obscure.

Subsequently, the CPU 227 determines whether the treatment for the subject is being continued by the operator (step S17). Specifically, the CPU 227 determines whether power is being supplied to the treatment instrument 301 by the treatment-instrument control device 302, and determines that the operator is continuing the treatment to the subject when the power is being supplied to the treatment instrument 301 by the treatment-instrument control device 302, and determines that the operator is not continuing the treatment to the subject when it is determined that the power is not being supplied to the treatment instrument 301 by the treatment-instrument control device 302. When the CPU determines that the treatment to the subject is being continued by the operator (step S17: YES), the endoscope control device 202 shifts to step S18 described later. On the other hand, when the CPU 227 determines that the treatment to the subject is not being continued by the operator (step S17: NO), the endoscope control device 202 ends this processing.

At step S18, the CPU 227 determines whether the devices of the respective components constituting the treatment system 1 has become an output OFF state. When it is determined that the devices of the respective components constituting the treatment system 1 has become the output OFF state by the CPU 227 (step S18: YES), the endoscope control device 202 ends this processing. On the other hand, when it is determined that the devices of the respective components constituting the treatment system 1 has not become the output OFF state (step S18: NO), the endoscope control device 202 returns to step S13 described above.

At step S19, the endoscope control device 202 performs haze-treatment control processing with respect to the haze in the field of view of the endoscope 201. Details of the haze-treatment control processing will be described later. After step S19, the endoscope control device 202 shifts to step S17.

### Details of Haze-Treatment Control Processing

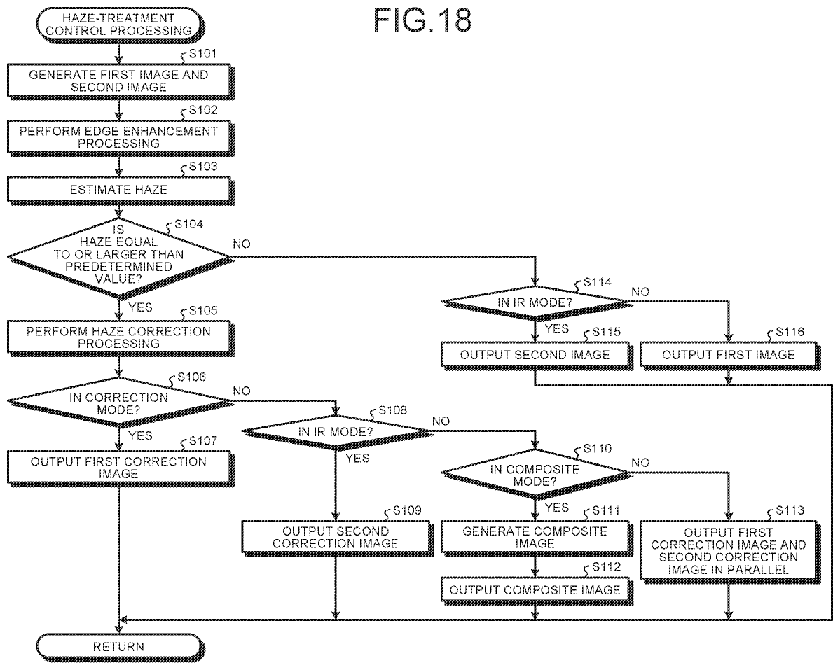

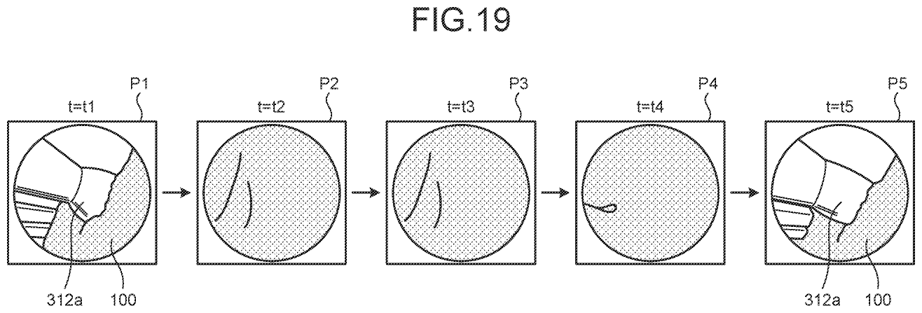

Next, details of the haze-treatment control processing explained at step S19 in FIG. 17 will be explained. FIG. 18 is a flowchart illustrating a detailed overview of the haze-treatment control processing in FIG. 17.

As illustrated in FIG. 18, first, the image processing unit 222 generates the first image and the second image (step S101). Specifically, the first-image generating unit 2222 generates the first image (color image by visible light) based on image data input from the image-data input unit 2221. Furthermore, the second-image generating unit 2224 generates the second image (IR image by invisible light) based on the image data input from the image-data input unit 2221.

Subsequently, the second correction-image generating unit 2227 performs publicly-known edge enhancement processing with respect to the second image (step S102). Specifically, the second correction-image generating unit 2227 performs edge extraction to extract a portion at which the brightness significantly changes with respect to the second image, and performs the edge enhancement processing to enhance the edge of the portion subjected to the edge extraction. The edge enhancement processing by the second correction-image generating unit 2227 may be performed by combining respective processing of, for example, publicly-known dilation processing, erosion processing, averaging processing, and median processing. Moreover, the edge extraction may be performed by combining, for example, one or more of known filters, such as the Sobel filter, the Laplacian filter, and the Canny filter.

Thereafter, the first detecting unit 2223 estimates a haze component of the field of view of the endoscope 201 based on the first image generated by the first-image generating unit 2222 (step S103). Specifically, the haze component in the field of view of the endoscope 201 is estimated by an estimation method similar to that of the haze estimating unit 2226a described above.

Subsequently, the haze determining unit 2230 determines whether the haze in the field of view of the endoscope 201 detected by the first detecting unit 2223 is equal to or larger than a predetermined value. When it is determined that the haze component in the field of view of the endoscope 201 detected by the first detecting unit 2223 is equal to or larger than the predetermined value by the haze determining unit 2230 (step S104: YES), the endoscope control device 202 shifts to step S105 described later. On the other hand, when it is determined that the haze component in the field of view of the endoscope 201 detected by the first detecting unit 2223 is not equal to or larger than the predetermined value by the haze determining unit 2230 (step S104: NO), the endoscope control device 202 shifts to step S114 described later.

At step S105, the first correction-image generating unit 2226 performs haze correction processing to remove or reduce the haze with respect to the first image. Specifically, first, the haze estimating unit 2226a estimates the haze component H(x, y) for the first image. Subsequently, the local-histogram generating unit 2226b determines a distribution of a histogram in a local region including the reference pixel of the first image and the neighboring pixels of the reference pixel based on the haze component H(x, y) input from the haze estimating unit 2226a. Thereafter, the statistical-information calculating unit 2226c calculates the representative brightness based on the statistical information of the brightness histogram input from the local-histogram generating unit 2226b. Subsequently, the correction-coefficient calculating unit 2226d calculates the correction coefficient to correct the contrast within the local region based on the haze component H(x, y) input from the haze estimating unit 2226a and the statistical information input from the statistical-information calculating unit 2226c. Finally, the contrast correcting unit 2226e performs the contrast correction of the reference pixel of the first image with respect to the first image input from the first-image generating unit 2222 based on the haze component H(x, y) input from the haze estimating unit 2226a and the correction coefficient input from the correction-coefficient calculating unit 2226d.

The image-processing control unit 2232 determines whether the display mode of the endoscope control device 202 is set to the correction mode of displaying an image in which the haze component is corrected (step S106). When it is determined that the display mode of the endoscope control device 202 is set to the correction mode of displaying an image in which the haze component is corrected by the image-processing control unit 2232 (step S106: YES), the endoscope control device 202 shifts to step S107 described later. On the other hand, when it is determined that the display mode of the endoscope control device 202 is not set to the correction mode of displaying an image in which the haze component is corrected by the image-processing control unit 2232 (step S106: NO), the endoscope control device 202 shifts to step S108 described later.

At step S107, the display-image generating unit 2229 generates the first correction image based on the first image for which the haze is corrected by the first correction-image generating unit 2226, to output to the display device 203. After step S107, the endoscope control device 202 returns to the main routine of the cutting procedure in FIG. 17 described above, and shifts to step S17.

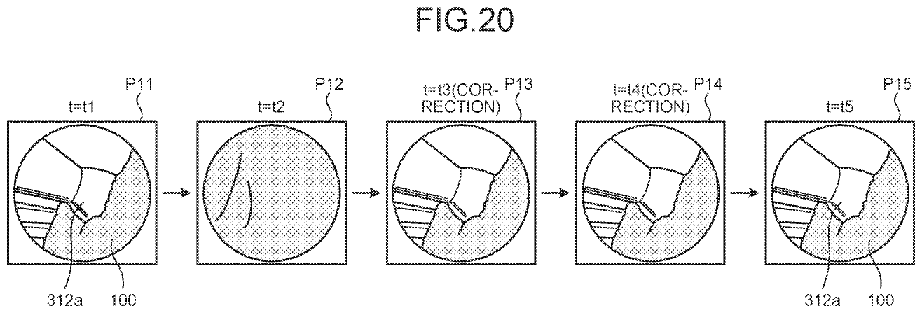

FIG. 19 is a diagram illustrating an example of the first image generated by the display-image generating unit 2229 based on the first image and output to the display device 203 when the haze correction processing by the first correction-image generating unit 2226 has not been performed. FIG. 20 is a diagram illustrating an example of the first correction image generated based on the first correction image by the display-image generating unit 2229 and output to the display device 203 when the haze correction processing by the first correction-image generating unit 2226 has been performed. Time axis in FIG. 19 and FIG. 20 is the same.

As indicated in a display image P1 to a display image P5 in FIG. 19, when the irrigation fluid becomes turbid due to dissolution of bone powder and the like into the irrigation fluid as a result of the treatment of the treatment target site 100 by the ultrasound transducer 312a of the ultrasound probe 312 in the field of view of the endoscope 201 (for example, refer to the display image P2 (time t=t2) to the display image P4 (time t=t4)), the operator cannot confirm a position of the ultrasound transducer 312a of the ultrasound probe 312 and the treatment target site 100 in the field of view of the endoscope 201, and a condition of cutting or the like by the ultrasound transducer 312a performed on the treatment target site 100.

On the other hand, as indicated in a first correction image P11 to a first correction image P15 in FIG. 20, when the irrigation fluid becomes turbid due to dissolution of bone powder and the like into the irrigation fluid as a result of the treatment of the treatment target site 100 by the ultrasound transducer 312a of the ultrasound probe 312, the display-image generating unit 2229 outputs to the display device 203 by using the first correction image from which the haze is reduced or removed by the first correction-image generating unit 2226 (for example, the first correction image P13 (time t=t3), the first correction image P14 (time t=4)). Thus, the operator can confirm the position of the ultrasound transducer 312a of the ultrasound probe 312 and the treatment target site 100 in the field of view of the endoscope 201, and a condition of cutting or the like by the ultrasound transducer 312a performed on the treatment target site 100 and, therefore, can perform the cutting procedure on the treatment target site 100 by the ultrasound probe 312 without interrupting.

Returning back to FIG. 18, step S108 and later will be explained.

At step S108, the image-processing control unit 2232 determines whether the display mode of the endoscope control device 202 is set to the IR mode of displaying the IR image, which is the second image. When it is determined that the display mode of the endoscope control device 202 is set to the IR mode of displaying the IR image, which is the second image by the image-processing control unit 2232 (step S108: YES), the endoscope control device 202 shifts to step S109 described later. On the other hand, when it is determined that the display mode of the endoscope control device 202 is not set to the IR mode of displaying the IR image, which is the second image, by the image-processing control unit 2232 (step S108: NO), the endoscope control device 202 shifts to step S110 described later.

At step S109, the display-image generating unit 2229 generates the second correction image, which is the IR image subjected to the edge enhancement, based on the second image generated by the second correction-image generating unit 2227, to output to the display device 203. After step S107, the endoscope control device 202 returns to the main routine of the cutting procedure in FIG. 17 described above, and shifts to step S17.

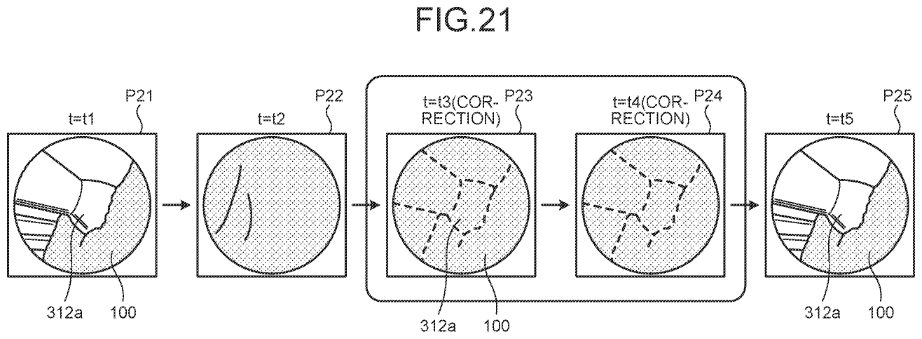

FIG. 21 is a diagram illustrating an example of the second correction image generated based on the second correction image by the display-image generating unit 2229 and output to the display device 203 when the edge enhancement processing by the second correction-image generating unit 2227 has been performed. The time axis of FIG. 21 is the same as the time axis of FIG. 19 described above.

As indicated in a second correction image P21 to a second correction image P25 in FIG. 21, the display-image generating unit 2229 outputs to the display device 203 by using the second correction image subjected to the edge enhancement processing to enhance respective outlines of the ultrasound transducer 312a of the ultrasound probe 312 and the treatment target site 100 by the second correction-image generating unit 2227 when the field of view of the endoscope 201 becomes cloudy as a result of treatment with respect to the treatment target site 100 by the ultrasound probe 312, the second correction-image generating unit 2227 outputs to the display device 203 by using the second correction image for which the edge enhancement processing to enhance the respective outlines of the ultrasound transducer 312a of the ultrasound probe 312 and the treatment target site 100 by the second correction-image generating unit 2227 (for example, the second correction image P23 (time t=t3), the second correction image P24 (time t=4)). Thus, the operator can view the ultrasound transducer 312a of the ultrasound probe 312 and the treatment target site 100 indirectly and, therefore, the cutting procedure on the treatment target site 100 by the ultrasound probe 312 can be performed without interrupting.

Returning back to FIG. 18, explanation of step S110 and later will be continued.

At step S110, the image-processing control unit 2232 determines whether the display mode of the endoscope control device 202 is set to the composite mode of displaying the composite image obtained by combining the first correction image and the second correction image. When it is determined that the display mode of the endoscope control device 202 is set to the composite mode of displaying the composite image obtained by combining the first correction image and the second correction image by the image-processing control unit 2232 (step S110: YES), the endoscope control device 202 shifts to step S111 described later. On the other hand, when it is determined that the display mode of the endoscope control device 202 is not set to the composite mode of displaying the composite image obtained by combining the first correction image and the second correction image by the image-processing control unit 2232 (step S110: NO), the endoscope control device 202 shifts to step S112 (parallel display mode) described later.

At step S111, the composite-image generating unit 2228 generates the composite image in which the first correction image generated by the first correction-image generating unit 2226 and the second correction image generated by the second correction-image generating unit 2227 are combined at a predetermined ratio, for example, 5:5.

Subsequently, the display-image generating unit 2229 outputs the composite image generated by the composite-image generating unit 2228 to the display device 203 (step S112). After step S112, the endoscope control device 202 returns to the main routine of the cutting procedure in FIG. 17 described above, and shifts to step S17.

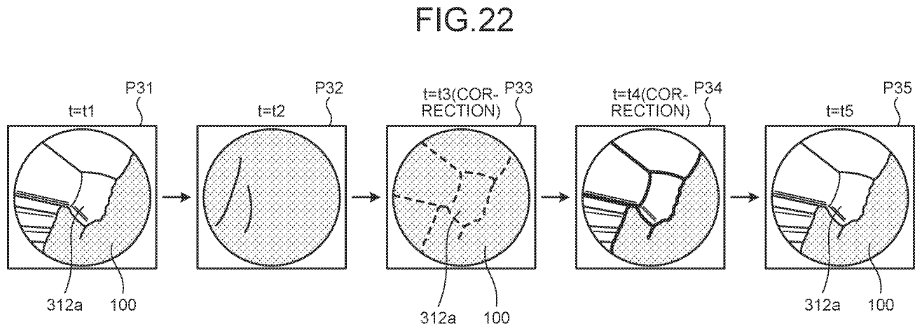

FIG. 22 is a diagram illustrating an example of the composite image generated based on the composite image by the display-image generating unit 2229 and output to the display device 203 when the composite processing by the composite-image generating unit 2228 is performed. The time axis of FIG. 22 is the same as the time axis of FIG. 19 described above.

As indicated in a composite image P31 to a composite image P35 in FIG. 22, when the field of view of the endoscope 201 becomes cloudy due to turbidity resulting from the procedure performed on the treatment target site 100 by the ultrasound probe 312, the display-image generating unit 2229 combines the first correction image from which the haze is reduced or removed by the first correction-image generating unit 2226 and the second correction image subjected to the edge enhancement processing to enhance the respective outlines of the ultrasound probe 312 and the treatment target site 100 by the second correction-image generating unit 2227, to output to the display device 203 (for example, the composite image P33 (time t=t3), the composite image P34 (time t=4)). Thus, the ultrasound transducer 312a of the ultrasound probe 312 and the treatment target site 100 are enhanced compared to other regions, and the operator can view the ultrasound transducer 312a of the ultrasound probe 312 and the treatment target site 100 easily and, therefore, can perform the cutting procedure on the treatment target site 100 by the ultrasound probe 312 without interrupting.

Returning back to FIG. 18, explanation of step S113 and later will be continued.

At step S113, the display-image generating unit 2229 outputs the first correction image generated by the first correction-image generating unit 2226 and the second correction image generated by the second correction-image generating unit 2227 in parallel to the display device 203. After step S113, the endoscope control device 202 returns to the main routine of the cutting procedure in FIG. 17 described above, and shifts to step S17.

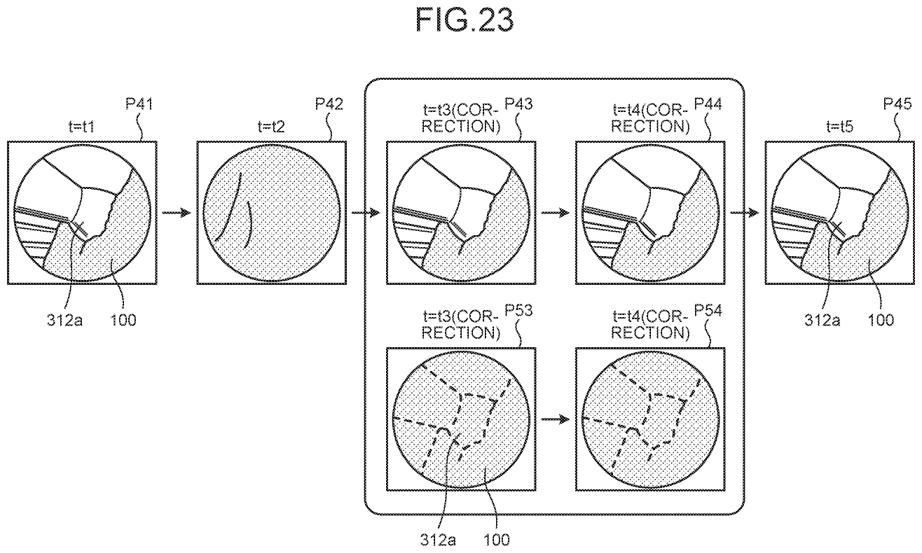

FIG. 23 is a diagram illustrating an example of images when the first correction image and the second correction image are output to the display device 203 by the display-image generating unit 2229. The time axis of FIG. 23 is the same as the time axis of FIG. 19 described above.

As illustrated in FIG. 23, when the field of view of the endoscope 201 has become cloudy as a result of the treatment of the treatment target site 100 by the ultrasound probe 312, the display-image generating unit 2229 outputs the first correction image generated by the first correction-image generating unit 2226 and the second correction image generated by the second correction-image generating unit 2227 in parallel to the display device 203 (for example, a first image P43, a second image P53 (time t=t3), a first image P44, a second image P54 (time t=t4)). thus, the operator can perform a procedure while comparing a state in which the haze has been removed and a state in which the ultrasound transducer 312a of the ultrasound probe 312 and the treatment target site 100 are enhanced.

At step S114, the image-processing control unit 2232 determines whether the display mode of the endoscope control device 202 is set to the IR mode of displaying the second image, which is an infrared image. When it is determined that the display mode of the endoscope control device 202 is set to the IR mode of displaying the second image, which is an infrared image by the image-processing control unit 2232 (step S114: YES), the endoscope control device 202 shifts to step S115 described later. On the other hand, when it is determined that the display mode of the endoscope control device 202 is not set to the IR mode of displaying the second image, which is an infrared image by the image-processing control unit 2232 (step S114: NO), the endoscope control device 202 shifts to step S116 described later.

At step S115, the display-image generating unit 2229 generates a display image by using the second image generated by the second-image generating unit 2224, to output to the display device 203. Thus, the operator can perform the procedure on the treatment target site 100 by using the ultrasound probe 312 while viewing the second image, which is an infrared image, displayed on the display device 203. After step S115, the endoscope control device 202 returns to the main routine of the cutting procedure in FIG. 17 described above, and shifts to step S17.

At step S116, the display-image generating unit 2229 generates a display image by using the first image generated by the first-image generating unit 2222, to output to the display device 203. Thus, the operator can perform the procedure on the treatment target site 100 by the ultrasound probe 312 while viewing the first image, which is a color image, displayed on the display device 203. After step S116, the endoscope control device 202 returns to the main routine of the cutting procedure in FIG. 17 described above, and shifts to step S17.

According to the first embodiment explained above, because the display-image generating unit 2229 generates a display image based on the first correction image input from the first correction-image generating unit 2226, to output to the display device 203, even when the field of view in the endoscope 201 is deteriorated, a treatment of the treatment target site 100 by using the treatment instrument 301 can be continued.

Moreover, according to the first embodiment, the display-image generating unit 2229 generates a display image based on the composite image input from the composite-image generating unit 2228, to output to the display device 203. As a result, the ultrasound transducer 312a of the ultrasound probe 312 and the treatment target site 100 are enhanced compared to other regions, and the operator can view the ultrasound transducer 312a of the ultrasound probe 312 and the treatment target site 100 easily and, therefore, can perform the cutting procedure on the treatment target site 100 by the ultrasound probe 312 without interrupting.

Furthermore, according to the first embodiment, the display-image generating unit 2229 generates a display image based on at least one of the first image input from the first-image generating unit 2222, the second image input from the second-image generating unit 2224, the first correction image input from the first correction-image generating unit 2226, the second correction image input from the second correction-image generating unit 2227, and the composite image input from the composite-image generating unit 2228 in accordance with the synchronization signal that is synchronized with the imaging drive of the imaging unit 204, to output to the display device 203. As a result, the operator can perform the cutting procedure on the treatment target site 100 by the ultrasound probe 312 without interrupting while viewing a smooth display image displayed on the display device 203.

Moreover, according to the first embodiment, when it is determined that the haze in the field of view of the endoscope 201 is equal to or larger than the predetermined value by the haze determining unit 2230, the display-image generating unit 2229 generates a display image based on the first correction image input from the first correction-image generating unit 2226, to output to the display device 203. On the other hand, when it is determined that the haze in the field of view of the endoscope 201 is not equal to or larger than the predetermined value by the haze determining unit 2230, the display-image generating unit 2229 generates a display image based on the first image generated by the first-image generating unit 2222, to output to the display device 203. Therefore, it is possible to display a normal display image (color image) until the field of view of the endoscope 201 becomes cloudy.

In the first embodiment, the second correction-image generating unit 2227 may generate the second correction-image data by subjecting the second image of infrared light to tone correction (for example, the edge enhancement processing) based on the haze detection result of the first image by the first detecting unit 2223, and the display-image generating unit 2229 may output a display image obtained by using the second correction-image data from the second correction-image generating unit 2227.

Moreover, in the first embodiment, the first correction-image generating unit 2226 may generate the first correction image data by subjecting the first image, which is a color image, to the tone correction (for example, the haze correction processing) based on the haze detection result of the second image by the second detecting unit 2225, and the display-image generating unit 2229 may output a display image obtained by using the first correction image from the first correction-image generating unit 2226 to the display device 203.

### Second Embodiment

Next, a second embodiment will be explained. In the first embodiment described above, the first image, which is a color image, and the second image, which is an IR image, are generated by the single imaging unit 204, but in the second embodiment, the first image, which is a color image, and the second image, which is an IR image, are generated by two imaging units. Specifically, in the second embodiment, a configuration of an endoscope is different. Therefore, in the following, an endoscope according to the second embodiment will be explained. Identical reference signs are assigned to identical components to those of the treatment system 1 according to the first embodiment, and detailed explanation will be omitted.

### Functional Configuration of Endoscope

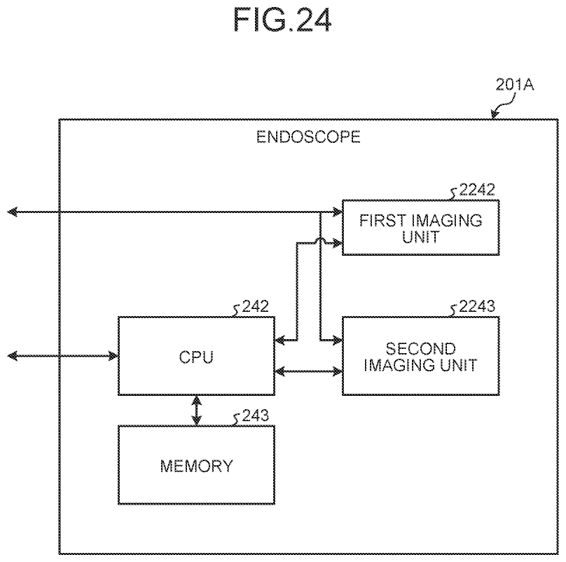

FIG. 24 is a block diagram illustrating a functional configuration of the endoscope according to the second embodiment.

An endoscope 201A illustrated in FIG. 24 includes a first imaging unit 2242 and a second imaging unit 2243 in place of the imaging unit 204 in the endoscope 201 according to the first embodiment described above.

The first imaging unit 2242 is constituted of multiple optical systems and an image sensor of either CCD or CMOS with a Bayer array color filter sensitive to visible light (wavelength band λ=380 nm to 780 nm) arranged on a light receiving surface. The first imaging unit 2242 generates the first image (PAW data from which the color first image data can be generated) by imaging a subject image formed by the optical system, and output this generated first image to the endoscope control device 202.

The second imaging unit 2243 is constituted of multiple optical systems and an image sensor of either CCD or CMOS with an IR filter sensitive to invisible light (wavelength band λ=780 nm to 2500 nm) arranged on a light receiving surface. The second imaging unit 2243 generates the second image (PAW data from which the second image data, which is IR data, can be generated) by imaging a subject image formed by the optical system, and outputs this second image to the endoscope control device 202.

In the cutting procedure using the endoscope 201A thus configured, the endoscope control device 202 performs similar processing to that in the cutting procedure according to the first embodiment described above. Therefore, detailed explanation of the cutting procedure using the endoscope 201A is omitted. Also for the cutting procedure using the endoscope 201A, the composite-image generating unit 2228 can generate a composite image.

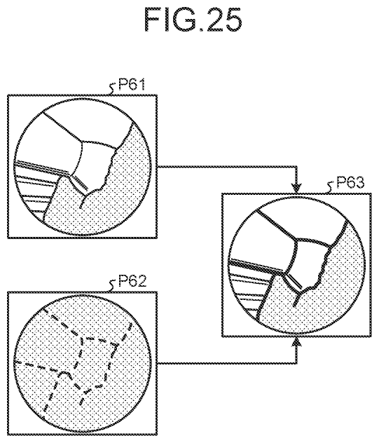

FIG. 25 is a diagram illustrating an example of the composite image generated by the composite-image generating unit 2228. As illustrated in FIG. 25, the composite-image generating unit 2228 generates a composite image P63 by combining a first correction image P61 that is the first image of a color image generated by the first imaging unit 2242, and from which a haze is reduced or removed by the first correction-image generating unit 2226 and a second correction image P62 that is the second image of an IR image generated by the second imaging unit 2243, and that is subjected to the edge enhancement processing by the second correction-image generating unit 2227 at a predetermined ratio.

The display-image generating unit 2229 outputs the composite image P63 generated by the composite-image generating unit 2228 to the display device 203.

Thus, the operator can easily view the ultrasound transducer 312a of the ultrasound probe 312 and the treatment target site 100 easily in a state in which the haze is removed or reduced and, therefore, can perform the cutting procedure on the treatment target site 100 by the ultrasound probe 312 without interrupting.

According to the second embodiment, an effect similar to that of the first embodiment described above is produced, and even when the field of view of the endoscope 201A is deteriorated, a treatment of the treatment target site 100 by using the treatment instrument 301 can be continued.

### Third Embodiment

Next, a third embodiment will be explained. In the first embodiment described above, the first illumination device 603 and the second illumination device 604 respectively irradiate visible light and invisible light to a subject, but in the third embodiment, light in a red wavelength bad, light in a green wavelength band, light in a blue wavelength band, and light in an infrared wavelength band are irradiated to a subject using a sequential method. Specifically, in the third embodiment, configurations of an endoscope and an illumination device are different. Accordingly, in the following, configurations of the endoscope and the illumination device according to the third embodiment will be explained. Identical reference symbols are assigned to identical components to those of the treatment system 1 according to the first embodiment described above, and detailed explanation thereof will be omitted.

### Functional Configuration of Endoscope

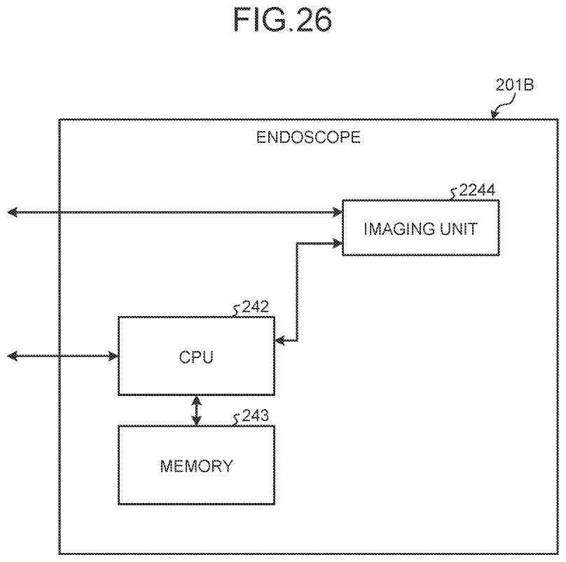

FIG. 26 is a block diagram illustrating a functional configuration of the endoscope according to the third embodiment.

An endoscope 201B illustrated in FIG. 26 includes an imaging unit 2244 in place of the imaging unit 204 in the endoscope 201 according to the first embodiment described above.

The imaging unit 2244 is constituted of multiple optical systems and an image sensor of either CCD or CMOS having pixels sensitive to visible light (wavelength band λ=400 nm to 680 nm) and invisible light (wavelength band λ=870 nm to 1080 nm). The imaging unit 2244 generates image data (RAW data) including a wavelength range of visible light or invisible light by imaging a subject image formed by the optical system, and outputs this generated image data to the endoscope control device 202.

### Functional Configuration of Illumination Device

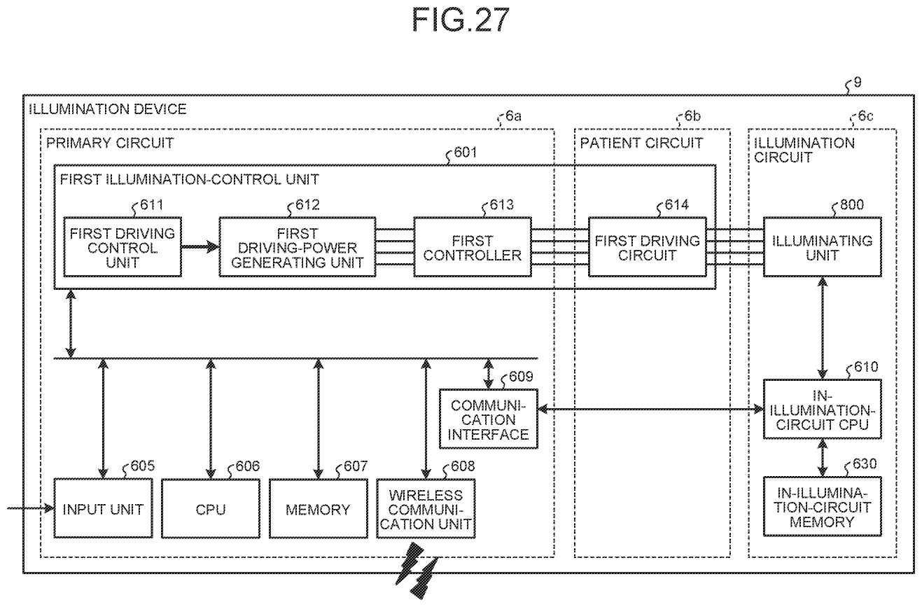

FIG. 27 is a block diagram illustrating a functional configuration of the illumination device according to the third embodiment.

The illumination device 9 illustrated in FIG. 27 excludes the second illumination device 604 and the second illumination-control unit 602 in the illumination device 6 according to the first embodiment described above, and includes an illuminating unit 800 in place of the first illumination device 603.

The illuminating unit 800 irradiates light of the red wavelength band, light of the green wavelength band, light of the blue wavelength band, and light of the infrared wavelength band to a subject by the sequential method under control of the first illumination-control unit 601 and the in-illumination-circuit CPU 610.

### Schematic Configuration of Illuminating Unit

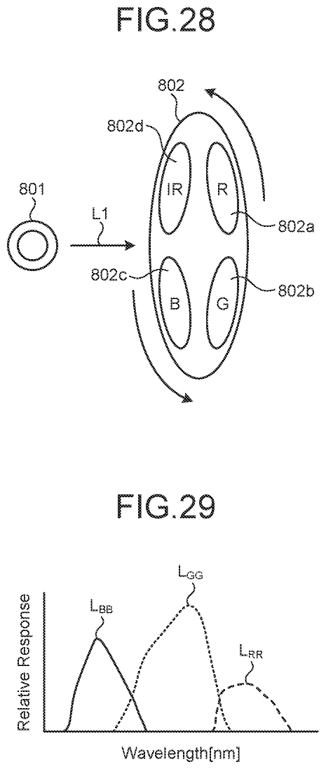

FIG. 28 is a schematic diagram illustrating a schematic configuration of the illuminating unit 800.

The illuminating unit 800 illustrated in FIG. 28 includes a light source 801 capable of emitting white light and a rotating filter 802 that is arranged in an optical path of the white light emitted by the light source 801 and that is rotated by a driving unit not illustrated.

The rotating filter 802 includes a red filter 802a that passes light of a red wavelength band, a green filter 802b that passes light of a green wavelength band, a blue filter 802c that passes light of a blue wavelength band, and an IR filter 802d that passes light of an infrared wavelength band. As the rotating filter 802 rotates, either one of the red filter 802a, the green filter 802b, the blue filter 802c, and the IR filter 802d is positioned on the optical path of the white light emitted by the light source 801.

FIG. 29 is a diagram illustrating a relationship between a transmission characteristic and wavelength band of the red filter 802a, the green filter 802b, and the blue filter 802c.

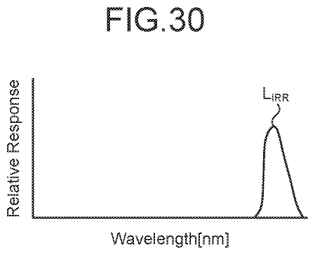

FIG. 30 is a diagram illustrating a relationship between a transmission characteristic and a wavelength band of the IR filter 802d.

In FIG. 29 and FIG. 30, a horizontal axis represents the wavelength and a vertical axis represents the transmittance. Moreover, in FIG. 29, a curve L_{RR} indicates the transmission characteristic of the red filter 802a, a curve L_{GG} indicates the transmission characteristic of the green filter 802b, and a curve L_{BB} indicates the transmission characteristic of the blue filter 802c. Furthermore, in FIG. 30, a curve L_{IRR} indicates the transmission characteristic of the IR filter 802d.

As illustrated in FIG. 29 and FIG. 30, the rotating filter 802 rotates, driven by a drive unit not illustrated, to transmit light in the red wavelength band, light in the green wavelength band, light in the blue wavelength band, and the light in the infrared wavelength band towards a subject.

In the cutting procedure using the illumination device 9 configured as described above, the endoscope control device 202 performs the same processing as that in the cutting procedure according to the first embodiment described above. Specifically, the endoscope control device 202 generates the first image, which is a color image, using the red image data, the green image data, and the blue image data that are generated as the imaging unit 2244 sequentially receives the light of the red wavelength band, the light of the green wavelength band, the light of the blue wavelength band, and the light of the infrared wavelength band, and the second image, which is an infrared image, using the infrared image data. In this case, the image processing unit 222 generates at least one of the first correction image and the second correction image, and the composite image using the first image and the second image, to output to the display device 203. Thus, an effect similar to that of the first embodiment described above is produced, and the operator can easily view the ultrasound transducer 312a of the ultrasound probe 312 and the treatment target site 100 easily in a state in which the haze is removed or reduced and, therefore, can perform the cutting procedure on the treatment target site 100 by the ultrasound probe 312 without interrupting.

According to the third embodiment explained above, an effect similar to that of the first embodiment described above is produced, and even when the field of view of the endoscope 201B is deteriorated, a treatment of the treatment target site 100 by using the treatment instrument 301 can be continued.

In the third embodiment, the light of the red wavelength band, the light of the green wavelength band, the light of the blue wavelength band, and the light of the infrared wavelength bad are irradiated to the subject by rotating the rotating filter 802, but, it is not limited thereto. For example, it may be configured using a red LED capable of emitting light of the red wavelength band, a green LED capable of emitting light of the green wavelength band, a blue LED capable of emitting light of the blue wavelength band, and an infrared LED capable of emitting light of the infrared wavelength band, and may irradiate light by sequentially causing the red LED, the green LED, the blue LED, and the infrared LED to emit light.

Moreover, in the third embodiment, a first rotating filter including an R filter, a G filter, and a B filter that pass light of the red wavelength band, the light of the green wavelength band, and the light of the blue wavelength band, respectively, and a second rotating filter including an IR filter that passes light of the infrared wavelength band may be provided, and it may be configured to position the first rotating filter or the second rotating filter on the optical path of the light source 801 according to the mode set to the endoscope control device 202, to be rotated.

Furthermore, in the third embodiment, a rotating filter that includes an R filter, a G filter, and a B filter that pass light of the red wavelength band, the light of the green wavelength band, and the light of the blue wavelength band, respectively, and a transparent filter, a first light source that is capable of emitting white light, and a second light source that is capable of emitting infrared light may be provided, and it may be configured to activate either one of the first light source and the second light source to emit light according to the mode set to the endoscope control device 202. Because the sequential method can increase the number of effective pixels of the imaging device, the resolution of a single pixel becomes high compared to the case in which a color filter is arranged on the imaging device, enabling the identification of finer bone particles.

Moreover, light is irradiated in the sequential method in the third embodiment, but it is not limited thereto, and light may be irradiated in a simultaneous method.

### Modifications of First to Third Embodiments

In the first to the third embodiments described above, the display-image generating unit 2229 switches an image to be output to the display device 203 according to the mode set to the endoscope control device 202, but it is not limited thereto. For example, the display-image generating unit 2229 may switch an image to be output to the display device 203 based on a driving signal and a synchronization signal (VT) of the treatment instrument 301 input from the treatment-instrument control device 302. Specifically, when either one of the driving signal to drive the treatment instrument 301 and the synchronization signal (VT) is input from the treatment-instrument control device 302, the display-image generating unit 2229 outputs at least one of the first correction image, the second correction image, and the composite image to the display device 203.

Because this enables to switch the content of the display image to be displayed on the display device 203 without changing the mode of the endoscope control device 202 each time, the operator can perform the cutting procedure on the treatment target site 100 using the ultrasound probe 312 without performing a complicated operation.

Furthermore, the display-image generating unit 2229 switches a type of image to be output to the display device 203 according to the synchronization signal, and the type of the image to be displayed on the display device 203 is switched smoothly and, therefore, it is possible to prevent discomfort for the operator and reduce burden on the operator.

### OTHER EMBODIMENTS

Moreover, in the first to the third embodiments of the present disclosure, it has been explained about the treatment for turbidity caused by bone powder in the irrigation fluid and the like, but it is not limited to those in fluids, and it can also be applied in air. In the first to the third embodiments, it can also be applied to the deterioration of visibility in the field of view of an endoscope caused by cutting debris, fat mist, and the like resulting from a procedure in air in a joint area.

Furthermore, in the first to the third embodiments of the present disclosure, it has been explained about the treatment of a knee joint, but it can be applied to other parts (spine or the like), not limited to the knee joint.

Moreover, the first to the third embodiments of the present disclosure can be applied to turbidity due to factors other than bone powder. For example, it can also be applied to debris, such as soft tissue, synovium, and fat, and other noise (cavitation, such as bubbles). For example, in the first to the third embodiments, as a factor of deterioration of the field of view caused by a treatment by the treatment instrument 301, it can also be applied to turbidity or visibility degradation caused by cutting debris of soft tissue such as cartilage, synovium, and fat as tissue fragments.

Furthermore, the first to the third embodiments of the present disclosure can be applied to the deterioration of visibility caused by fine bubbles resulting from factors, such as cavitation associated with ultrasound vibrations of the treatment instrument 301 in a procedure in liquid using the treatment instrument 301.

Moreover, the first to the third embodiments of the present disclosure can be applied even when the field of view of the endoscope 201 is obstructed by a relatively large tissue fragment. In this case, the endoscope control device 202 may be configured to determine whether the field of view of the endoscope 201 is obstructed by an obstruction based on the first image, and to perform image processing to remove the obstruction by using a publicly-known technique when it is determined to be obstructed by an obstruction. In this case, the endoscope control device 202 may perform the image processing using the size of a treatment area by the treatment instrument 301, a duration for which the treatment target site 100 is occluded, and the like to the extent that it does not affect the processing.

Furthermore, the first to the third embodiments of the present disclosure can be applied also when a filter that passes near-infrared light (700 nm to 2500 nm) or a LED that is capable of emitting near-infrared light is used, instead of infrared light.

Moreover, in the first to the third embodiments of the present disclosure, the composite-image generating unit 2228 may generate a composite image in which the second correction image and the first image are combined, or may generate a composite image by combining the second correction image and the first correction image. Furthermore, by combining the multiple components disclosed in the treatment system according to the first to the third embodiment of the present disclosure, various embodiments can be formed. For example, from the entire components described in the treatment system according to the first to the third embodiment of the present disclosure described above, some of the components may be removed. Furthermore, the components explained in the treatment system according to the first to the third embodiment of the present disclosure described above may be combined as appropriate.

Moreover, in the treatment system according to the first to the third embodiment of the present disclosure, “unit” that has been used in the above description can be replaced with “means”, “circuit”, or the like. For example, the control unit may be referred to as control means or control circuit.

Furthermore, a program that is executed by the treatment system according to the first to the third embodiments of the present disclosure is stored in a computer-readable storage medium, such as a compact disk read-only memory (CD-ROM), a flexible disk (FD), a compact disk rewritable (CD-R), a digital versatile disk (DVD), a USB medium, and a flash memory, in a form of file data in an installable format or in an executable format, to be provided.

Moreover, a program executed by the treatment system according to the first to the third embodiments of the present disclosure may be stored in a computer that is connected through a network, such as the Internet, to be provided by being downloaded through the network.

Furthermore, in the explanation of flowcharts in the present specification, expressions such as “first”, “thereafter”, “subsequently”, and the like are used to indicate the sequence of processing between steps. However, the order of processing to implement the disclosure is not uniquely determined by these expressions. That is, the sequence of processing in the flowcharts described in the present specification can be changed within a range not causing contradictions. Moreover, not limited to a program constituted of simple branching processing as described, more judgment criteria may be determined to branch.

According to the present disclosure, an effect is produced that a procedure on a treatment site can be continued even when the field of view of an endoscope is deteriorated.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising a processor comprising hardware, the processor being configured to:
acquire first image data partially including a region in which a living body is treated with at least an energy treatment instrument;
detect a change in tone from at least a part of a region of a first image corresponding to the first image data to obtain a first detection result;
perform tone correction on the first image based on the first detection result to generate first correction-image data; and
generate a display image based on the first correction-image data.

2. The image processing device according to claim 1, wherein
the processor is further configured to:
acquire second image data that has a different wavelength band from the first image, and that partially includes the region;
detect a change in tone from at least a part of a region of a second image corresponding to the second image data to obtain a second detection result; and
perform tone correction on the second image based on the second detection result to generate second correction-image data, and
the display image is generated based on at least one of the first correction-image data and the second correction-image data.

3. The image processing device according to claim 2, wherein
the processor is further configured to combine the first correction-image data and the second correction-image data to generate composite image data, and
the display image is generated based on the composite image data.

4. The image processing device according to claim 2, wherein
the processor is further configured to:
generate a first display image based on the first correction-image data,
generate a second display image based on the second correction-image data, and
output at least one of the first display image and the second display image to a display.

5. The image processing device according to claim 1, wherein
the processor is further configured to generate the display image based on a synchronization signal that is synchronized with imaging drive of an imager.

6. The image processing device according to claim 1, wherein
the processor is further configured to perform the tone correction on the first correction-image data based on a driving signal of the energy treatment instrument to treat the living body and the first detection result to generate the first correction-image data.

7. The image processing device according to claim 1, wherein
the processor is further configured to detect a haze included in the first image as the change in tone.

8. The image processing device according to claim 7, wherein
the processor is further configured to:
determine whether the detected haze is equal to or larger than a predetermined value;
generate the display image based on the first correction-image data when it is unit determined that the detected haze is equal to or larger than the predetermined value; and
generate the display image based on the first image data when it is determined that the detected haze is not equal to or larger than the predetermined value.

9. The image processing device according to claim 1, wherein
the processor is further configured to:
estimate a haze component of each pixel in the first image;
generate a brightness histogram based on the first image and the estimated haze component;
calculate a representative brightness based on the generated brightness histogram;
calculate a correction coefficient to correct a contrast of the first image based on the estimated haze component and the calculated representative brightness; and
perform contrast correction of a reference pixel with respect to the first image based on the estimated haze component and the calculated correction coefficient to generate the first correction-image data.

10. The image processing device according to claim 2, wherein
the processor is further configured to acquire image data generated by an imager that is capable of receiving invisible light at least including an infrared wavelength band as the second image data.

11. The image processing device according to claim 10, wherein
the processor is further configured to perform edge enhancement processing with respect to the second image to generate the second correction-image data.

12. The image processing device according to claim 11, wherein
the processor is further configured to:
estimate a haze component of each pixel in the first image;
generate a brightness histogram based on the first image and the estimated haze component;
calculate a representative brightness based on the generated brightness histogram;
calculate a correction coefficient to correct a contrast of the first image based on the estimated haze component and the calculated representative brightness; and
perform contrast correction of a reference pixel with respect to the first image based on the estimated haze component and the calculated correction coefficient to generate the first correction-image data.

13. The image processing device according to claim 12, wherein
the processor is further configured to combine the first correction-image data and the second correction-image data to generate a composite image, and
the display image is generated based on the composite image data.

14. The image processing device according to claim 1, wherein
the processor is further configured to acquire image data generated by an imager that is capable of receiving visible light having a wavelength band inside a visible spectrum and invisible light having a wavelength band outside the visible spectrum as the first image data.

15. The image processing device according to claim 2, wherein
the processor is further configured to:
acquire image data generated by a first imager that is capable of receiving visible light having a wavelength band inside a visible spectrum as the first image data; and
acquire image data generated by a second imager that is capable of receiving invisible light having a wavelength band outside the visible spectrum as the second image data.

16. The image processing device according to claim 1, wherein
the processor is further configured to:
acquire second image data having a different wavelength from the first image;
detect a change in tone from at least a part of a region of a second image corresponding to the second image data to obtain a second detection result; and
perform tone correction on the second image based on the second detection result to generate second correction-image data, and
the display image is generated based on at least one of the first correction-image data and the second correction-image data.

17. An image processing device comprising a processor comprising hardware, the processor being configured to:
acquire a first image corresponding to first image data that includes a region in which a living body is treated with an energy treatment instrument;
acquire a second image corresponding to second image data having a different wavelength from the first image;
detect a change in tone from at least a part of a region of the first image to obtain a detection result;
perform tone correction on the second image based on the detection result to generate correction image data; and
generate a display image based on the correction image data.

18. A treatment system comprising:
an energy treatment instrument that can be inserted into a subject, and that is capable of treating a treatment target site;
an endoscope that can be inserted into the subject, and that is capable of generating first image data by imaging at least the treatment target site; and
an image processing device that performs image processing with respect to the first image data to output the processed first image data to a display,
the image processing device comprising a processor comprising hardware, the processor being configured to:
acquire the first image data;
detect a change in tone from at least a part of a region of a first image corresponding to the first image data to obtain a first detection result;
perform tone correction on the first image based on the first detection result to generate first correction-image data; and
generate a display image based on the first correction-image data.

19. An image processing method that is performed by an image processing device included in a processor comprising hardware, the method comprising:
acquiring first image data that includes a region in which a living body is treated with an energy treatment instrument;
detecting a change in tone from at least a part of a region of a first image corresponding to the first image data to obtain a first detection result;
performing tone correction on the first image based on the first detection result to generate first correction-image data; and
generating a display image based on the first correction-image data.
